(19) United States
(12) Patent Application Publication
(10) Pub. No.: US 2008/0319042 A1
(43) Pub. Date: Dec. 25, 2008

(54) Compositions and methods for treatment of liver disease

(76) Inventor: Harry Fong, Glen Ellyn, IL (US)

Correspondence Address: TROUTMAN SANDERS LLP, 600 PEACHTREE STREET , NE, ATLANTA, GA, 30308, US

(21) Appl. No.: 12/069,905

(22) Filed: Feb. 13, 2008

Related U.S. Application Data
(60) Provisional application No. 60/901,602, filed on Feb. 13, 2007.
(60) Provisional application No. 61/000,550, filed on Oct. 26, 2007.

(51) Int. Cl. A61K 31/407; A61K 31/35; C07D 311/94; A61P 1/16; C07D 519/00; A61K 31/352

(52) U.S. Cl. 514/411; 514/456; 514/455; 549/396; 549/386; 548/431

(57) ABSTRACT

The present invention comprises compositions and methods for the treatment of liver disease, particularly treatments for infection by HCV and/or HBV. The present invention comprises antiviral compositions comprising lamiridosin, derivatives of lamiridosin or iridoids that are effective in inhibiting one or more steps in the infection of cells by HCV or HBV. Methods for treating subjects, particularly humans, infected with HCV or HBV are provided.

### RELATED APPLICATIONS

This Application claims the priority of U.S. Provisional Patent Application Nos. 60/901,602, filed Feb. 13, 2007, and 61/000,550, filed Oct. 26, 2007, each of which is incorporated herein in its entirety.

### TECHNICAL FIELD

The present invention is related to methods and compositions for treating liver disease, particularly resulting from viral infection such as hepatitis C virus infection and the sequelae associated therewith.

### BACKGROUND OF THE INVENTION

Approximately 4 million persons in the United States are infected with the hepatitis C virus (HCV), a virus transmitted by blood-to-blood contact that can lead to liver damage and liver cancer. Hepatitis C is one of five currently identified viruses—Hepatitis A, B, C, D, and E—all of which can attack and damage the liver. Widely viewed as one of the most serious of the five, the hepatitis C Virus (HCV) is spread primarily through contact with infected blood and can cause cirrhosis (irreversible and potentially fatal liver scarring), liver cancer, or liver failure. Hepatitis C is the major reason for liver transplants in the United States, accounting for thousands of the procedures annually. The disease is responsible for between 8,000-10,000 deaths yearly in the United States.

Estimates are that the number of HCV-infected people may be four times the number of those infected with HIV, the AIDS virus. Presently, there is no vaccine or other means of preventing Hepatitis C infection. HCV exists in many different forms, called genotypes, confounding researchers in their quest to develop a vaccine effective for all variations. Also, HCV mutates frequently within infected patients, so even if an effective vaccine is developed, it could be rendered useless by a new strain of mutant virus.

Once HCV is contracted, enhancement of the immune response is effective for a small portion of patients. In most others, HCV's frequent mutations allow it to evade the immune system, defeating attempts to develop a cure. There are treatments available, but they don't work for all patients. One of the few approved treatments for chronic hepatitis C, especially for patients with consistently elevated liver enzymes or mild to moderate liver damage is alpha-interferon. Hepatitis C patients must inject interferon themselves, usually three times a week. In about 25% of patients, the drug has a pronounced effect in reducing HCV to a very low level in the blood. However, if the drug is ineffective after three months, it is generally discontinued.

A combination product of interferon with the antiviral drug Ribavirin is also approved. The interferon that is given is pegylated alpha-interferon and is given in combination with Ribavirin. Because of its ease of administration and better efficacy, peg-interferon has been replacing standard interferon both as monotherapy and as combination therapy for hepatitis C. Ribavirin is an oral antiviral agent that alone has little effect on HCV but by adding it to peg-interferon, it increases the sustained response rate by 2 to 3 fold. The combination therapy appears to suppress blood levels of HCV more effectively than a first or repeat course of Interferon alone. The combined therapy comes with side effects such as depression, increased risk of suicide, psychoses, extreme fatigue, restlessness at night, fetal death, malformations and other side effects that make it unusable by many patients. Currently, chronic hepatitis C patients who do not respond to therapy have few options. In many, cirrhosis and other damage will eventually cause the liver to stop functioning. In these cases, a liver transplant is the only recourse. However, even new livers may become infected with the virus.

A complicating factor for treatment is that patients are often infected with multiple viruses such as HIV, or patients have alcohol and other drug problems, and are immunocompromised or are in nutritionally compromised conditions. These patients are hard to treat and hepatitis C treatment is much less likely to be effective.

Few options exist for patients who either do not respond to therapy or who respond and later relapse. Patients who relapse after a course of Interferon monotherapy may respond to a course of combination therapy, particularly if they become and remain HCV or RNA negative during the period of monotherapy. The response rates in optimal dose and duration of peg-interferon and Ribavirin for relapse or previous non-responder patients have not been defined. An experimental approach to treatment for non-responders is the use of long-term or maintenance Interferon, which is feasible only if the peg-interferon is well tolerated and has a clear cut effect on serum aminotransferases or liver histology, despite lack of clearance of HCV RNA. This approach is now under evaluation.

One alternative for non-responders is a low weekly dose of peg-interferon-alpha 2B with the anti-inflammatory drug colchicine. Colchicine showed early promise as a treatment for advanced liver disease, but now studies suggest it does not slow the disease progression. Conventional treatments have shown sustained benefit in approximately 55% of patients. Because of the side effects, many patients turn to other alternative treatments. Side effects include flu like symptoms, body aches, fever, chills, fatigue, nausea and other gastrointestinal problems; hair loss, emotional changes, skin reactions and in more severe cases, depression, organ damage, blood conditions and other problems.

What is needed are compositions and methods for treating humans infected with hepatitis C that are effective. Additionally, treatment of other liver diseases, such as diseases of the liver caused by other viruses, such as Hepatitis B, or other causes, are also needed. It would be beneficial if such compositions and methods did not cause deleterious side effects.

### SUMMARY

The present invention is directed to compositions and methods for treating infections due to HCV, hepatitis C virus, and HBV, hepatitis B virus. The compositions comprise pharmaceutical compositions comprising novel compounds, lamiridosin and derivatives thereof. Compositions comprise iridoids and derivatives thereof. Methods of the present invention comprise treating HCV infection and inhibiting HCV replication. Compositions of the present invention are effective in inhibiting at least a step in the infection process of HCV and/or HBV.

Compositions of the present invention comprise lamiridosin, which is the aglycone form of lamiridoside. Lamiridosin is a compound having the structure

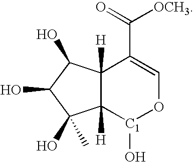

Compositions comprise derivatives of lamiridosin, or iridoids or derivatives of iridoids.

The invention comprises novel compounds, which are derivatives of lamiridosin,

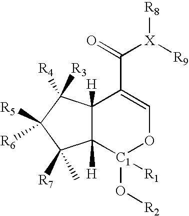

wherein R₁ is hydrogen, or R and OR₁ taken together can form an oxo group;
R₂ is hydrogen, β-D-glucopyranosyl group, (C₁₋₁₀)alkyl, (C₂₋₁₀)alkenyl, (C₂₋₁₀)alkynyl, or any of which groups can be optionally substituted with one to twenty-two of the same or different halogen atoms;
R₃ and R₄ are independently hydrogen, —OR′, —OC(O)R″, halo, R₃ and R₄ together can form an oxo group, and R′ and R″ are as described as below;
R₅ and R₆ are independently hydrogen, —OR′, —OC(O)R″, halo, R₅ and R₆ together can form an oxo group, and R′ and R″ are as described as below;
R₇ is hydrogen or hydroxy;
R₈ and R₉ are independently hydrogen, (C₁₋₁₀)alkyl, (C₂₋₁₀)alkenyl, (C₂₋₁₀)alkynyl, or any of which groups can be optionally substituted with one to twenty-two of the same or different halogen atoms;
R′ is hydrogen, (C₁₋₁₀)alkyl, (C₂₋₁₀)alkenyl, (C₂₋₁₀)alkynyl, or any of which groups can be optionally substituted with one to twenty-two of the same or different halogen atoms;
R″ is hydrogen, (C₁₋₁₀)alkyl, (C₂₋₁₀)alkenyl, (C₂₋₁₀)alkynyl, (C₃₋₁₀)cycloalkyl, (C₈₋₁₄)bicycloalkyl, (C₈₋₁₄)tricycloalkyl, (C₄₋₁₀)cycloalkenyl, (C₈₋₁₄)bicycloalkenyl, (C₈₋₁₄)tricycloalkenyl, aryl, substituted aryl, (3- to 6-membered)heterocycle, or any of which groups can be optionally substituted with one to twenty-two of the same or different halogen atoms; and
X is oxygen or nitrogen. Novel compounds that are derivatives of lamiridosin comprise

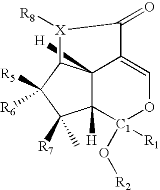

wherein R₁ is hydrogen, or R and OR₁ taken together can form an oxo group;
R₂ is hydrogen, β-D-glucopyranosyl group, (C₁₋₁₀)alkyl, (C₂₋₁₀)alkenyl, (C₂₋₁₀)alkynyl, or any of which groups can be optionally substituted with one to twenty-two of the same or different halogen atoms;
R₅ and R₆ are independently hydrogen, —OR′, —OC(O)R″, halo, R₅ and R₆ together can form an oxo group, and R′ and R″ are as described as below;
R₈ is hydrogen, (C₁₋₁₀)alkyl, (C₂₋₁₀)alkenyl, (C₂₋₁₀)alkynyl, or any of which groups can be optionally substituted with one to twenty-two of the same or different halogen atoms;
X is oxygen or nitrogen;
R′ is hydrogen, (C₁₋₁₀)alkyl, (C₂₋₁₀)alkenyl, (C₂₋₁₀)alkynyl, or any of which groups can be optionally substituted with one to twenty-two of the same or different halogen atoms;
R″ is hydrogen, (C₁₋₁₀)alkyl, (C₂₋₁₀)alkenyl, (C₂₋₁₀)alkynyl, (C₃₋₁₀)cycloalkyl, (C₈₋₁₄)bicycloalkyl, (C₈₋₄)tricycloalkyl, (C₄₋₁₀)cycloalkenyl, (C₈₋₁₄)bicycloalkenyl, (C₈₋₁₄)tricycloalkenyl, aryl, substituted aryl, (3- to 6-membered)heterocycle, or any of which groups can be optionally substituted with one to twenty-two of the same or different halogen atoms.

The invention comprises methods of treatment of humans or animals infected with HCV or HBV comprising administering an effective amount of a pharmaceutically acceptable formulation of at least one of the compounds taught herein. Methods for making compounds taught herein are also comprised in the invention.

### DESCRIPTION OF FIGURES

FIG. 1 is a bar graph showing anti-HVC activity of isolates.

FIG. 2 is a graph of dose-response of lamiridosin in anti-HVC assay (determination of IC₅₀).

FIG. 3 is a bar graph showing viral entry blocking activity of lamiridosin and other compounds.

FIG. 4 is a bar graph showing viral entry blocking activity by lamiridosin for several viruses.

FIGS. 5A-H are graphs showing the activity of particular compounds.

### DETAILED DESCRIPTION

The present invention comprises compositions and methods for treating liver disease, particularly infections due to HCV, hepatitis C virus, and HBV, hepatitis B virus. Compositions comprise lamiridosin and derivatives thereof, such as those shown in Table 5. Compositions may comprise iridoid compounds and derivatives thereof. Compositions may comprise combinations and mixtures of iridoids and lamiridosin and may be used in the methods for treatment of HCV or HBV. Methods of the present invention comprise treating liver diseases, including HCV infection and inhibiting HCV replication, by administering compositions of the present invention. Methods of the present invention comprise treating liver diseases, including HBV infection and inhibiting HBV replication, by administering compositions of the present invention.

The present invention comprises compositions comprising lamiridosin, for example lamiridosin which has been extracted from the plant Lamium album, or lamiridosin derivatives. The present invention discloses treatments for HCV and/or HBV using lamiridosin or its derivatives. Examples of lamiridosin derivatives are shown in Table 5. The invention is useful in both clinical and scientific research applications.

As used herein, the term “lamiridosin” refers to the active form of lamiridoside, the aglycone of lamiridoside, as represented in Formula (I). This compound may be a naturally extracted compound or a synthesized compound. In one embodiment, a method of the present invention for treatment of infection by HCV or HBV comprises administration of a composition comprising lamiridosin or its derivatives, or a combination or mixture of lamiridosin and one or more lamiridosin derivatives. Compositions may further comprises other active agents, such as steroids, antibiotics, antivirals, or combinations of two or more of these.

In another aspect, the present invention comprises pharmaceutical compositions comprising lamiridosin or its derivatives and a pharmaceutically acceptable vehicle. In an embodiment, the pharmaceutically acceptable vehicle comprises a carrier. In another embodiment, the pharmaceutically acceptable vehicle comprises an inactive vehicle such as an excipient. The present invention also provides kits containing a pharmaceutical composition of the present invention.

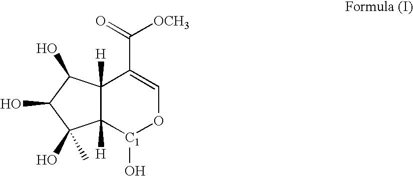

Certain compounds of the present invention, such as those of Formula I or its derivatives, such as those in Table 5, may have asymmetric centers and therefore exist in different enantiomeric and diastereomeric forms. Such compounds can be in the form of an optical isomer or a diastereomer. Accordingly, the invention encompasses the compound of Formula (I) and Table 5 and their uses as described herein in the form of their optical isomers, diasteriomers and mixtures thereof, including a racemic mixture.

One or more hydrogen, carbon or other atoms of a compound of Formula (I) or a derivative can be replaced by an isotope of the hydrogen, carbon or other atoms. Such compounds, which are encompassed by the present invention, are useful as research and diagnostic tools in metabolism pharmacokinetic studies and in binding assays.

In another embodiment, the lamiridosin composition can be prepared by mixing the lamiridosin with one or more pharmaceutically acceptable vehicles, such as carriers, excipients, auxiliaries and/or diluents.

Furthermore, the present invention provides pharmaceutical compositions comprising the compounds of the present invention and additionally, pharmaceutically active agents together with one or more pharmaceutically acceptable vehicles. The present invention also provides pharmaceutical compositions comprising the compounds of the present invention, and kits comprising pharmaceutical compositions of the present invention. Pharmaceutically active agents include, but are not limited to, antiviral compounds such as interferon or pegylated interferon, amantadine, rimantadine, gancyclovir, acyclovir, ribavirin, penciclovir, oseltamivir, foscamet zidovudine (AZT), didanosine (ddI), lamivudine (3TC), zalcitabine (ddC), stavudine (d4T), nevirapine, delavirdine, indinavir, ritonavir, vidarabine, nelfinavir, saquinavir, relenza, tamiflu, pleconaril, interferons, etc. as well as an antibody which immunospecifically binds to a target virus), steroids and corticosteroids such as prednisone, cortisone, fluticasone and glucocorticoid, antibiotics, analgesics, bronchodilators, or other treatments for viral infections.

The present invention comprises compositions comprising known iridoids for the treatment of HCV infection in humans and other animals. Such compounds may be used in the treatment of HCV infection in humans and other animals. See Table 7 for examples of such iridoids. Compositions comprise compounds including iridoid glycosides designated by the structural formulae (V)-(XIX), and iridoid aglycones designated by the structural formulae (XX)-(XXXIII), and pharmaceutically accepted salts, or combinations of iridoids glycosides and aglycones, or combinations of two or more iridoids glycosides, or combinations of two or more iridoids aglycones. Such compositions are used in methods for the inhibition of hepatitis C virus replication, or other steps in the replication cycle or infection. In the formulae (V)-(XIX), Glc refers to β-D-glucopyranosyl group, and in the formulae (XX)-(XXXIII), the configuration of C₁ is either S (Sinister) or R (Rectus). See Table 7.

Iridoids represent a group of monoterpenoid compounds and are found usually as glycosides in nature. When they are subjected to enzymatic hydrolysis by a sugar hydrolyzing enzyme such as β-glucosidase, a glucose molecule and an aglycone are formed as the hydrolyzed products. The aglycone form may be converted to its dialdehyde products and it may proceed to further conversion as shown in the following scheme.

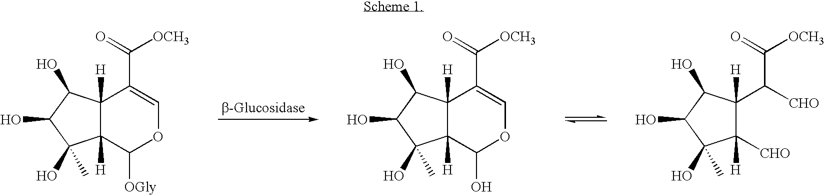

Though not wishing to be bound by any particular theory, it is currently thought that the aglycone iridoids compounds may show more biological activity than the glycoside iridoid compounds.

The present invention comprises compositions comprising at least one iridoids compound, glycoside or aglycone, for the treatment of HCV infection in humans. The present invention comprises compositions comprising at least one iridoids compound, glycoside or aglycone, for the treatment of HBV infection in humans. In an aspect, the present invention comprises pharmaceutical compositions comprising at least one of the iridoids compounds of Table 7 and a pharmaceutically acceptable vehicle. In an embodiment, the pharmaceutically acceptable vehicle comprises a carrier. In another embodiment, the pharmaceutically acceptable vehicle comprises an inactive vehicle such as an excipient. The present invention also provides kits containing a pharmaceutical composition of the present invention.

Certain compounds of the present invention, such as those of Table 7 or derivatives or those compounds may have asymmetric centers and therefore exist in different enantiomeric and diastereomeric forms. Such compounds can be in the form of an optical isomer or a diastereomer. Accordingly, the invention encompasses the compounds of Table 7 and their uses as described herein in the form of their optical isomers, diasteriomers and mixtures thereof, including a racemic mixture.

One or more hydrogen, carbon or other atoms of a compound of at least one of the iridoids compounds of Table 7 can be replaced by an isotope of the hydrogen, carbon or other atoms. Such compounds, which are encompassed by the present invention, are useful as research and diagnostic tools in metabolism pharmacokinetic studies and in binding assays.

In another embodiment, the iridoid composition can be prepared by mixing at least one of the iridoids compounds of Table 7 with one or more pharmaceutically acceptable vehicles, such as carriers, excipients, auxiliaries and/or diluents.

Furthermore, the present invention provides pharmaceutical compositions comprising the iridoid compounds of the present invention and additionally, pharmaceutically active agents together with one or more pharmaceutically acceptable vehicles. The present invention also provides pharmaceutical compositions comprising the compounds of the present invention, and kits comprising pharmaceutical compositions of the present invention. Pharmaceutically active agents include, but are not limited to, antiviral compounds such as interferon or pegylated interferon, amantadine, rimantadine, gancyclovir, acyclovir, ribavirin, penciclovir, oseltamivir, foscamet zidovudine (AZT), didanosine (ddI), lamivudine (3TC), zalcitabine (ddC), stavudine (d4T), nevirapine, delavirdine, indinavir, ritonavir, vidarabine, nelfinavir, saquinavir, relenza, tamiflu, pleconaril, interferons, etc. as well as an antibody which immunospecifically binds to a target virus), steroids and corticosteroids such as prednisone, cortisone, fluticasone and glucocorticoid, antibiotics, analgesics, bronchodilators, or other treatments for viral infections.

The methods of the present invention relate to treatment of a disease associated with a virus, including but not limited to, HCV and HBV, which infect humans, and may infect other animals. In another embodiment, the methods of the present invention comprise the use of lamiridosin and/or its derivatives, examples of which are shown in Table 5, and use of iridoids compounds as shown in Table 7 and/or pharmaceutically acceptable salts for treatment of HCV or HBV.

The present invention comprises methods for treating, ameliorating, managing, or preventing diseases caused by HCV or HBV, by administering at least one of the iridoids compounds of Table 7 or its derivatives and/or pharmaceutically acceptable salts alone or in combination with pharmaceutically active agents.

The present invention comprises treatment, amelioration, management and prevention of disease associated with infection by HCV or HBV, that affects animals, which include at least human. In specific embodiments, the virus is HCV and/or HBV. The invention also relates to administration of at least one of the iridoids compounds of Table 7 intravenously, rectally, parenterally, enterally, transdermally, via feeding tubes, and topically.

In another embodiment, the methods of the present invention comprise the use of at least one of the iridoids compounds of Table 7 and/or pharmaceutically acceptable salts. Whether a particular treatment of the invention is effective to treat HCV or HBV can be determined by any method known in the art.

The safety and efficiency of the proposed method of treatment may be tested in the course of systematic medical and biological assays on animals, toxicological analyses for acute and systemic toxicity, histological studies and functional examinations, and clinical evaluation of patients having a variety of indications for HCV or HBV.

The efficacy of the method of the present invention may be tested in appropriate animal models, and in human clinical trials, by any method known in the art. For example, the animal or human subject may be evaluated for any indicator of HCV or HBV that the method of the present invention is intended to treat. The efficacy of the method of the present invention for treatment of HCV or HBV can be assessed by measuring the levels of nucleic acid molecules, proteins of HCV or HBV in the animal model or human subject at suitable time intervals before, during, or after treatment. Any change or absence of change can be identified and correlated with the effect of the treatment on the subject.

The invention provides methods for the identification of a compound that inhibits the ability of HCV or HBV to infect a host or a host cell. In certain embodiments, the invention provides methods for the identification of a compound such as lamiridosin, its derivatives of Table 5, or at least one of the iridoid compounds of Table 7 that reduce the ability of HCV or HBV to replicate in a host or a host cell, reduces or inhibits the ability of HCV or HBV to enter a host or host cell, or other measurements of activity of HCV or HBV and its inhibition that are associated with infection by HCV or HBV. Any technique well-known to the skilled artisan can be used to screen for a lamiridosin derivative that would abolish or reduce the ability of HCV or HBV to infect a host and/or to replicate in a host or a host cell.

In certain embodiments, the invention comprises methods for the identification of a compound that inhibits the ability of HCV or HBV to replicate in a mammal or a mammalian cell, such as lamiridosin, its derivatives of Table 5, or at least one of the iridoid compounds of Table 7. The invention comprises methods for the identification of a compound that inhibits the ability of HCV or HBV to infect a mammal or a mammalian cell. In certain embodiments, the invention comprises methods for the identification of a compound that inhibits the ability of HCV or HBV to replicate in a mammalian cell. In a specific embodiment, the mammalian cell is a human cell.

In another embodiment, a cell is contacted with a compound such as lamiridosin, its derivatives of Table 5, or at least one of the iridoid compounds of Table 7 and infected with HCV or HBV. In certain embodiments, a control culture is infected with the HCV or HBV in the absence of the compounds of the present invention. The cell can be contacted with the compound or composition of the present invention before, concurrently with, or subsequent to the infection with HCV or HBV. In a specific embodiment, the cell is a mammalian cell, or the cell is a human cell. In certain embodiments, the cell is incubated with the compound or composition for at least 1 minute, at least 5 minutes, at least 15 minutes, at least 30 minutes, at least 1 hour, at least 2 hours, at least 5 hours, at least 12 hours, or at least 1 day. The titer of the virus or the measurement of viral activity can be measured at any time during the assay. In certain embodiments, a time course of viral growth in the culture is determined. If the viral growth is inhibited or reduced in the presence of the test compound, the compound is identified as being effective in inhibiting or reducing the growth or infection of HCV or HBV. In a specific embodiment, the compound that inhibits or reduces the growth of HCV or HBV is tested for its ability to inhibit or reduce the growth rate of other viruses to test its specificity for HCV or HBV.

In an embodiment, a compound such as lamiridosin, its derivatives of Table 5, or at least one of the iridoid compounds of Table 7, or a composition of the present invention is administered to an animal and the animal is infected with HCV or HBV. In certain embodiments, a control animal is infected with HCV or HBV without the administration of the compound or composition. The compound or composition can be administered before, concurrently with, or subsequent to the infection with HCV or HBV. In a specific embodiment, the animal is a mammal or other animal that can serve as a host for HCV or HBV. The titer of the virus in the model animal can be measured at any time during the assay. In certain embodiments, a time course of viral growth in the culture is determined. If the viral growth is inhibited or reduced in the presence of the compound or composition, the compound or composition is identified as being effective in inhibiting or reducing the growth or infection of HCV or HBV. In a specific embodiment, the compound that inhibits or reduces the growth of HCV or HBV in the animal is tested for its ability to inhibit or reduce the growth rate of other viruses to test its specificity for HCV or HBV.

Efficacy and dose of the therapeutic agents of the present invention can be determined as by methods known to those skilled in the art. Assays for antiviral activity are disclosed herein and may include a viral neutralization tests or plaque or foci reduction assays. The procedure used for in-vitro antiviral susceptibility testing is as follows: the in vitro susceptibility against HCV or HBV is performed in 96-well microtitre plates seeded with appropriate cells. Two-fold dilutions of antiviral agents starting from more than 4 times the peak serum concentration after the maximum therapeutic dose to less than one-quarter of the trough serum concentration are tested in quadruplicate against 100 TCID50 of HCV or HBV. A corresponding set of cell controls with compound or composition but without virus inoculation should be used as controls for toxicity.

The cells are then scored for the inhibition of the resulting cytopathic effect (CPE) or other measurement of viral activity. Their antiviral activities may also be compared in other cell lines. Those likely to have clinically significant inhibitory activity can be tested by the plaque reduction assay.

For the plaque reduction assay, 24-well tissue culture plates are prepared with a confluent cell monolayer (1×10⁵ cells per well) in an appropriate media, such as 1.0 ml of minimal essential medium (MEM) with 10% fetal calf serum (FCS). After the medium is aspirated, 50-100 plaque forming units (PFU) of HCV can be added to each well. Plates are incubated for an appropriate amount of time for infection and entry of the virus, such as 2 hours at 37° C. in 5% CO₂. The inoculum is aspirated and 1.0 ml of overlay (1.0% low-melting point agarose in 1% FCS/MEM with corresponding drug dilutions) added to each well. Plates are further incubated for an appropriate time, such as 48 hours at 37° C. in 5% CO₂. Cells are then fixed by adding 2 ml of 10% formaldehyde and incubating the plates at room temperature for 2 hours. The agarose plugs are then aspirated, and each well stained with 0.5% crystal violet prepared in 70% methanol. After destaining wells with several washes of water, the viral plaques are then counted. There are other well known methods for testing viral activity and its inhibition by compounds of the present invention, and such methods are contemplated by the present invention.

Methods of the present invention comprise administering an effective amount of a composition comprising at least one compound such as lamiridosin, its derivatives of Table 5, or at least one of the iridoid compounds of Table 7 to a human or animal infected with HCV. Methods of the present invention comprise administering an effective amount of a composition comprising at least one compound such as lamiridosin, its derivatives of Table 5, or at least one of the iridoid compounds of Table 7 to a human or animal infected with HBV. Various delivery systems are known and can be used to administer the pharmaceutical compositions of the invention, e.g., encapsulation in liposomes, microparticles or microcapsules. Methods of administration of the compositions of the present invention include but are not limited to intradermal, intramuscular, intraperitoneal, intravenous, subcutaneous, intranasal, epidural, and oral routes. The compounds or compositions may be administered by any convenient route, for example by infusion or bolus injection, by absorption through epithelial or mucocutaneous linings (e.g., oral mucosa, rectal and intestinal mucosa, etc.) and may be administered together with other pharmaceutically active agents. Administration can be systemic or local.

In a specific embodiment, it may be desirable to administer the pharmaceutical compositions of the present invention locally to the area in need of treatment; this may be achieved by, for example, and not by way of limitation, local infusion during surgery, topical application, e.g., dermal patches, by injection, by means of a catheter, by means of a suppository, or by means of an implant, said implant being of a porous, non porous, or gelatinous material, including membranes, such as silastic membranes, or fibers. In one embodiment, administration can be by direct injection at the site (or former site) infected tissues.

In another embodiment, the pharmaceutical composition can be delivered in a vesicle, in particular a liposome. Liposome technology is well known in the art, and those of skill can readily formulate liposomal delivery vehicles for the compounds of the present invention.

In yet another embodiment, the pharmaceutical compositions can be delivered in a controlled release system. In one embodiment, a pump may be used to administer compositions of the present invention. In another embodiment, polymeric materials can be used. Other controlled release systems are known to those skilled in the art.

The pharmaceutical compositions of the present invention comprise a therapeutically or prophylactically effective amount of at least one compound such as lamiridosin, its derivatives of Table 5, or at least one of the iridoid compounds of Table 7 and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the pharmaceutical composition is administered. Such pharmaceutical carriers can be sterile liquids, such as water and oils, including those of petroleum, animal, vegetable or synthetic origin, such as peanut oil, soybean oil, mineral oil, sesame oil and the like.

Water is a suitable carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. The formulation should suit the mode of administration.

In an embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lidocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

In an embodiment, the composition is formulated in accordance with routine procedures as a pharmaceutical composition adapted for oral administration to human beings. Oral delivery formulations are known in the pharmaceutical arts and where necessary, the composition may also include other components for oral delivery.

The pharmaceutical compositions of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2 ethylamino ethanol, histidine, procaine, etc.

The amount of the pharmaceutical composition of the invention which will be effective in the prevention (i.e., a prophylactically effective amount) and/or treatment (i.e., a therapeutically effective amount) of a particular disorder or condition will depend on the nature of the disorder or condition, and can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. However, suitable dosage ranges for intravenous administration are generally about 0.01 mg to about 15 mg of active compound per kilogram body weight. Suitable dosage ranges for intranasal administration are generally about 0.01 pg/kg body weight to 1 mg/kg body weight. In an embodiment, the dose is at least 100, 200, 300, 400, 500, 600, 700, 1000 mg per person per treatment. The course of treatment may be once every 3, 12, 24, 36, 48 hours. The complete treatment cycle may be for 2, 4, 6, 8, 10, 14, 18, 20, 30 days. Effective doses may be extrapolated from dose response curves derived from in vitro or animal model test systems.

Suppositories generally contain active ingredient in the range of 0.5% to 10% by weight; oral formulations preferably contain 10% to 95% active ingredient.

The invention also provides a pharmaceutical pack or kit comprising one or more containers filled with one or more of the compounds of the present invention such as lamiridosin, its derivatives of Table 5, or at least one of the iridoid compounds of Table 7 in a pharmaceutical composition of the invention. Optionally associated with such container(s) can be a notice in the form prescribed by a governmental agency regulating the manufacture, use or sale of pharmaceuticals or biological products, which notice reflects approval by the agency of manufacture, use or sale for human administration. In a preferred embodiment, the kit contains an anti-viral agent of the invention, e.g., lamiridosin and/or its derivatives, alone or in combination with adjuvants, antivirals, antibiotics, analgesic, or other pharmaceutically acceptable excipients.

The present invention further encompasses kits comprising a container containing a pharmaceutical composition of the present invention and instructions for use.

The efficacy of the treatment methods of the present invention may be evaluated by measuring the levels of HCV or HBVand also other criteria, such as viral DNA or RNA, viral antigens, or antibodies to viral antigens for the clinical diagnosis of the subject. A method for detecting the presence or absence of HCV or HBV involves obtaining a biological sample from various sources and contacting the sample with a compound or an agent capable of detecting an epitope or nucleic acid of HCV or HBV such that the presence of HCV or HBV is detected in the sample.

An assay for determining the efficacy of compounds of the present invention in therapeutic methods for treating diseases associated with HCV or HBV may be based on a determination of whether a lamiridosin derivative compound or one of the iridoids compounds of Table 7 has the same binding capability or affinity, or a similar level of antiviral activity as lamiridosin, or the lamiridosin derivative compound or one of the iridoids compounds of Table 7 has more or less effectiveness in inhibiting HCV or HBV as lamiridosin for treatment, prevention, amelioration or management of HCV or HBV symptoms. In certain embodiments, the lamiridosin derivative compounds or the iridoids compounds of Table 7 that are useful in the present method of invention have a similar dissociation constant, total or partial ionic charge or charges. Many assays may be used to determine the efficacy of a lamiridosin derivative compound or one of the iridoids compounds of Table 7.

The aglycone form, lamirodosin, and lamiridoside were initially isolated from Lamium album. A method of isolation comprises the following steps. In brief, the dry, milled Lamium album plant material (74.2 g) was extracted with MeOH (5×500 ml) to yield a crude extract (11.732 g). The MeOH extract was absorbed in 17.431 g Si gel (230-400 mesh, Natland International Cooperation) and chromatographed over a Si gel (112.8 g, 230-400 mesh, Natland International Cooperation) column, eluting with CHCl₃/Me₂CO/MeOH to afford 23 fractions. The fraction F019 (325 mg) was subjected to preparative HPLC separation [Phenomenex, LUNA 10μ C18(2), 10 μM, 250×50 mm], eluting with MeOH—H₂O 30:70 with a flow rate of 12 ml/min to afford lamiridoside (F30, 97.22 mg). Lamiridoside was also detected in the fractions F18, F20 and F21. HPLC analysis showed that lamiridoside (Formula II) (Glc refers to β-D-glucopyranosyl group) is a major component of the MeOH extract of the Lamium album plant material (ca. 0.4%).

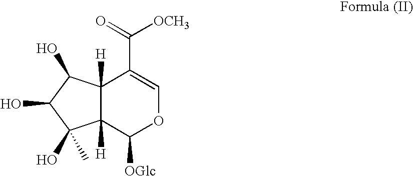

The major component was obtained as a colorless gum. Anomeric signals of a sugar unit were observed in its 1H and 13C NMR spectra [δ 4.60 (1H, d, J=7.91 Hz) and δ 99.72 (d)], and the NMR signals of the sugar unit correspond to those of β-D-glucopyranosyl group. The aglycone of the compound was determined to be an iridoid monoterpene by comparison of its NMR data with those of known iridoids. The NMR data of the compound was found to be identical to those of lamiridoside reported by Eigtved P, et al. from the same plant species (1974). A major component in the Lamium album plant material was determined to be lamiridoside. The corresponding aglycone is an inseparable pair of epimers obtained by enzyme hydrolysis, and is referred to herein as lamiridosins A/B or lamiridosin, and the terms may be used interchangeably.

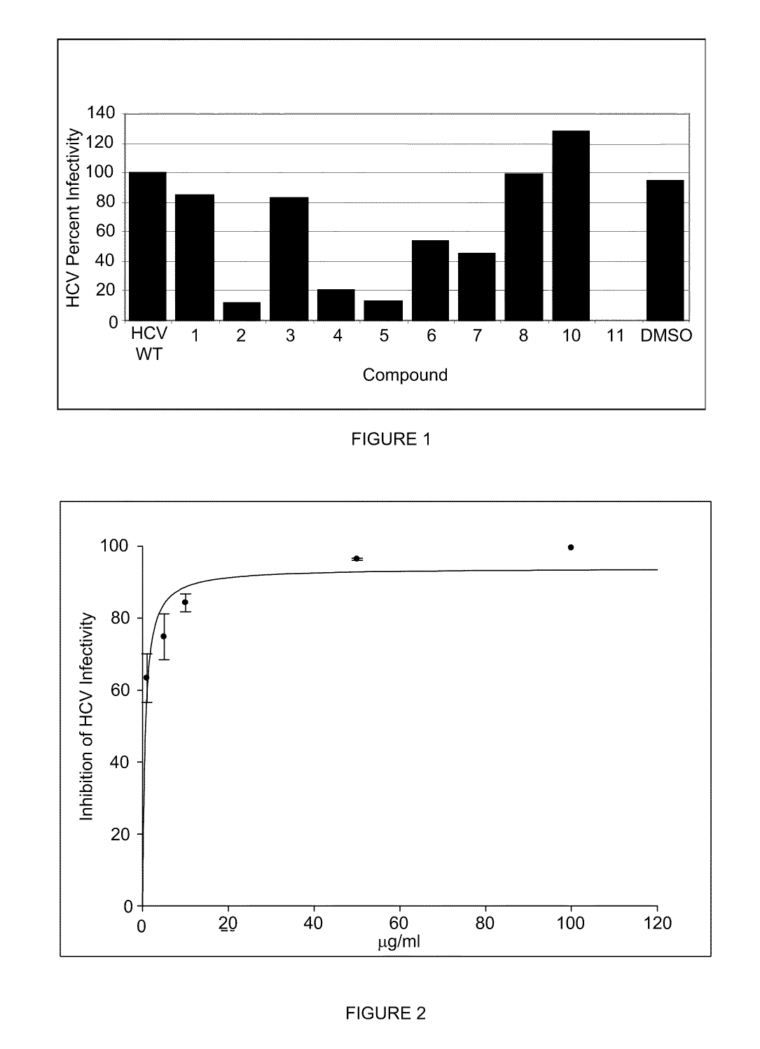

Evaluation of the iridoid glycoside lamiridoside for anti-hepatitis B virus activity (anti-HBSAg and Anti-HBV DNA) (Tables 1-3) and anti-hepatitis C virus activity (FIG. 1) assays showed it to be inactive in all three bioassays. For comparison purposes, aucubin and its anti-HBV DNA active aglycone were employed as reference controls. In FIG. 1, samples were tested for their ability to inhibit HCV/HIV entry at a concentration of 100 μg/mL. The data show that samples #2 (lamiridosin), #4 (aucubin aglycone), and #5 gave approximately 80% inhibition; samples #6 and #7 show about 50% inhibition; and samples #1 (lamiridoside), #3 (aucubin), #8, and #10 are inactive. Sample #11 was toxic to the host HUH7 cells. Evaluation of lamiridosin in the anti-HBSAg assay showed it to be inactive at doses of 37.5 and 50 μg/ml (Tables 1 and 2). However, lamiridosin inhibited DNA expression by 44.5% at a concentration of 37.5 μg/ml in the anti-HBV DNA assay (Table 3).

Lamiridosin exerted a direct inhibition on the infectivity of HCV (Anti-HCV) ca. 85% at a dose of 100 μg/ml, see FIG. 1. The IC₅₀ of lamiridosin in the anti-HCV assay was found to be 0.6 μg/ml (2.31 μmol) (FIG. 2 and Table 4). The data in FIG. 2 were fit to a simple IC₅₀ binding isotherm. The resulting fitted parameters are shown in the plot. The result clearly indicates that lamiridosin inhibits HCV viral entry with an IC₅₀ value of 0.6 μg/mL. This compound was non-cytotoxic to the Hep G2 2.2.15 cells at doses greater than 50 μg/ml. The Imax of lamiridosin was 93.9±3.5%. FIG. 2 shows a dose response curve for lamiridosin in an anti-HCV assay.

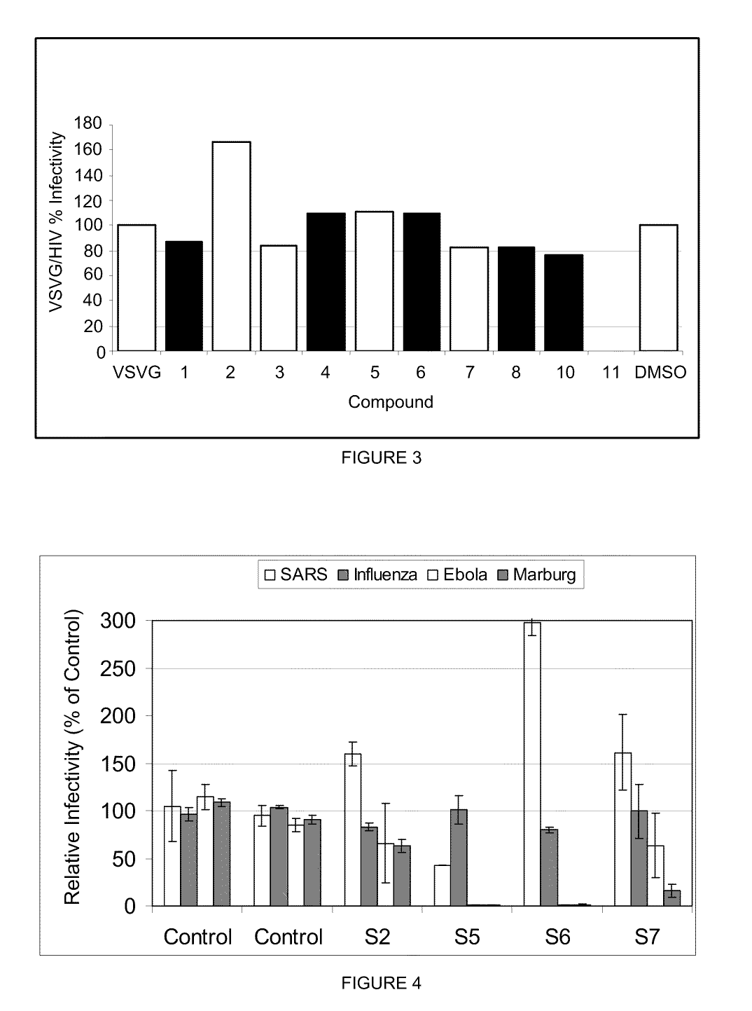

To determine the specificity of lamiridosin toward HCV, and not against other viruses, its ability to block the entry of the VSVG/HIV (vesicular stomtatis virus G protein) pseudovirions into HUH7 cells was tested. The VSVG virus has more promiscuous proteins that can bind to receptors and can infect multiple cell types. In FIG. 3, samples were tested for the ability to inhibit VSVG/HIV pseudoviral entry at a concentration of 100 μg/mL. The results in FIG. 3 show that lamiridosin does not block VSVG viral entry, but did block HCV viral entry (FIG. 1), which indicates that this compound has anti-viral activity against the hepatitis C virus. In FIG. 3, 1 is lamiridoside, 2 is lamiridosin, 3 is aucubin, and 4 is aucubin aglycone.

The present invention comprises compositions comprising derivatives of lamiridosins, and such compositions may be used for the treatment of liver disease, including inhibiting the replication of HCV and HCV. Examples of lamiridosin derivatives of the present invention are shown in Table 5. Derivatives of lamiridosin may have the generic structure (III) or (IV),

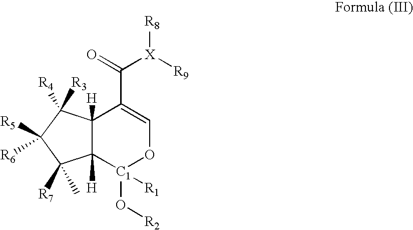

wherein R₁ is hydrogen, or R and OR₁ taken together can form an oxo group;
R₂ is hydrogen, β-D-glucopyranosyl group, (C₁₋₁₀)alkyl, (C₂₋₁₀)alkenyl, (C₂₋₁₀)alkynyl, or any of which groups can be optionally substituted with one to twenty-two of the same or different halogen atoms;
R₃ and R₄ are independently hydrogen, —OR′, —OC(O)R″, halo, R₃ and R₄ together can form an oxo group, and R′ and R″ are as described as below;
R₅ and R₆ are independently hydrogen, —OR′, —OC(O)R″, halo, R₅ and R₆ together can form an oxo group, and R′ and R″ are as described as below;
R₇ is hydrogen or hydroxy;
R₈ and R₉ are independently hydrogen, (C₁₋₁₀)alkyl, (C₂₋₁₀)alkenyl, (C₂₋₁₀)alkynyl, or any of which groups can be optionally substituted with one to twenty-two of the same or different halogen atoms;
R′ is hydrogen, (C₁₋₁₀)alkyl, (C₂₋₁₀)alkenyl, (C₂₋₁₀)alkynyl, or any of which groups can be optionally substituted with one to twenty-two of the same or different halogen atoms;
R″ is hydrogen, (C₁₋₁₀)alkyl, (C₂₋₁₀)alkenyl, (C₂₋₁₀)alkynyl, (C₃₋₁₀)cycloalkyl, (C₈₋₁₄)bicycloalkyl, (C₈₋₁₄)tricycloalkyl, (C₄₋₁₀)cycloalkenyl, (C₈₋₁₄)bicycloalkenyl, (C₈₋₁₄)tricycloalkenyl, aryl, substituted aryl, (3- to 6-membered)heterocycle, or any of which groups can be optionally substituted with one to twenty-two of the same or different halogen atoms;
X is oxygen or nitrogen;
The configuration of C₁ is either S (Sinister) or R (Rectus).

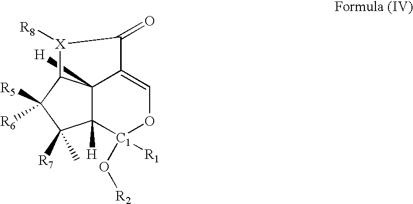

wherein R₁ is hydrogen, or R and OR₁ taken together can form an oxo group;
R₂ is hydrogen, β-D-glucopyranosyl group, (C₁₋₁₀)alkyl, (C₂₋₁₀)alkenyl, (C₂₋₁₀)alkynyl, or any of which groups can be optionally substituted with one to twenty-two of the same or different halogen atoms;
R₅ and R₆ are independently hydrogen, —OR′, —OC(O)R″, halo, R₅ and R₆ together can form an oxo group, and R′ and R″ are as described as below;
R₈ is hydrogen, (C₁₋₁₀)alkyl, (C₂₋₁₀)alkenyl, (C₂₋₁₀)alkynyl, or any of which groups can be optionally substituted with one to twenty-two of the same or different halogen atoms;
X is oxygen or nitrogen;
R′ is hydrogen, (C₁₋₁₀)alkyl, (C₂₋₁₀)alkenyl, (C₂₋₁₀)alkynyl, or any of which groups can be optionally substituted with one to twenty-two of the same or different halogen atoms; R″ is hydrogen, (C₁₋₁₀)alkyl, (C₂₋₁₀)alkenyl, (C₂₋₁₀)alkynyl, (C₃₋₁₀)cycloalkyl, (C₈₋₁₄)bicycloalkyl, (C₈₋₁₄)tricycloalkyl, (C₄₋₁₀)cycloalkenyl, (C₈₋₁₄)bicycloalkenyl, (C₈₋₁₄)tricycloalkenyl, aryl, substituted aryl, (3- to 6-membered)heterocycle, or any of which groups can be optionally substituted with one to twenty-two of the same or different halogen atoms;
The configuration of C₁ is either S (Sinister) or R (Rectus).

Methods of synthesizing such derivatives include, but are not limited to, the following:

The reaction protocol of derivatives (III) and (IV) is exemplified in Scheme 1 with lamiridosin as the starting material. The C₁ hydroxyl group of lamiridosin is methylated for protection of this group, and the resulting compound D1 is then hydrolyzed with 2N NaOH to afford compound D2. D2 is lactonized by 1,3-dicyclohexylcarbodiimide (DCC) in CH₂Cl₂ to afford compound D3, which is followed by esterification with a selected acyl chloride reagent to yield the desired products D4. Oxidation of D3 by treatment with pyridinium chlorochromate (PCC) in CH₂Cl₂ affords D5, which is followed by base hydrolyzation with 2N NaOH to yield D6. D6 is esterified with a selected acyl chloride reagent to yield another desired products D7. Compound D7 further couples with 1-hydroxybenztriazole (HOBT) in the presence of DCC to yield D8, which reacts with different amines to furnish yet another group of desired products D9. In addition, lamiridosin reacts directly with a selected acyl chloride reagent to afford the desired products D10. The lactone D5 is treated with the trimethylaluminum (AlMe₃) and with different primary amines to afford an intermediate, which is further treated with triphenylphosphine (TPP) and diethylazodicarboxylate (DEAD) in toluene to yield the desired products D11.

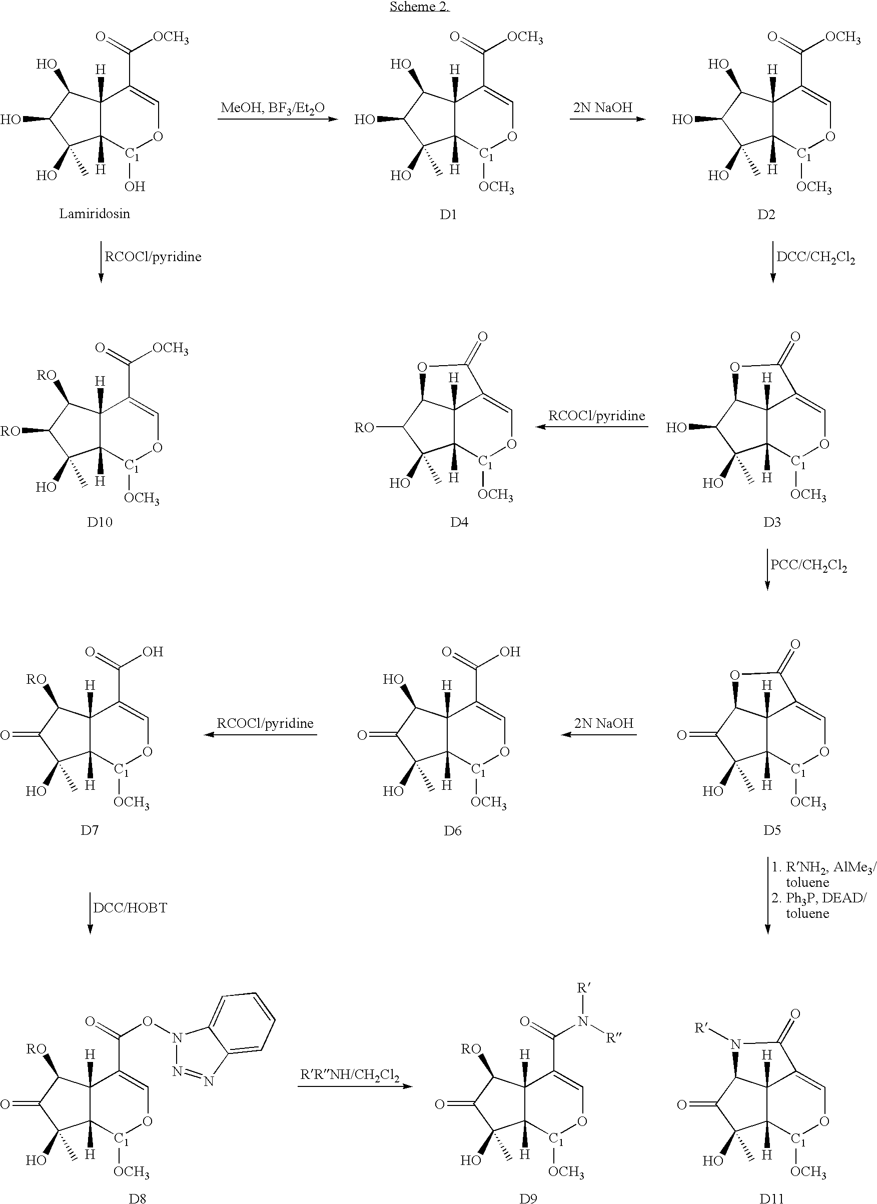

### DEFINITIONS

As used herein, the term “analog” in the context of a non-proteinaceous analog refers to a second organic or inorganic molecule which possesses a similar or identical function as a first organic or inorganic molecule and is structurally similar to the first organic or inorganic molecule.

As used herein, the term “derivative” in the context of a non-proteinaceous derivative refers to a second organic or inorganic molecule that is formed based upon the structure of a first organic or inorganic molecule. A derivative of an organic molecule includes, but is not limited to, a molecule modified, e.g., by the addition or deletion of a hydroxyl, methyl, ethyl, carboxyl, halogen or amine group. An organic molecule may also be esterified, alkylated, cyclized and/or phosphorylated.

As used herein, the term “pharmaceutically active agent” refers to any medically useful substance, including any therapeutically beneficial substance which may be used in combination with the lamiridosin or its derivative or composition of the present invention, including, but not limited to: viricides; microbicides; antibiotics; amino acids; peptides; vitamins; co-factors for protein synthesis; hormones including growth hormones; endocrine tissue; living cells including for example: stem cells, chondrocytes, bone marrow cells, and parenchymal cells; synthesizers; enzymes; biocompatible surface active agents; antigenic agents; growth factors including but not limited to: transforming growth factor, and insulin like growth factor; immunosuppressants; and permeation enhancers.

As used herein, the terms “subject” and “patient” are used interchangeably. As used herein, the terms “subject” and “subjects” refer to an animal, preferably a mammal including a non-primate (e.g., cows, pigs, horses, goats, sheep, cats, dogs, ducks, ferrets, ferret badgers, rabbits, raccoon dogs, civets, avian species and rodents), a primate, and a human.

As used herein, the term “variant” refers either to a naturally occurring genetic mutant of hepatitis B or hepatitis C virus or a recombinantly prepared variation of hepatitis B or hepatitis C virus.

As used herein, the term “vehicle” refers to a substance that facilitates the use of a drug, pigment, or other material mixed with it. The New Oxford American Dictionary (2001).

As used herein, the term “carrier” refers to a substance used to support or convey another substance such as a pigment, catalyst, or radioactive material. The New Oxford American Dictionary (2001).

As used herein, the term “excipient” refers to an inactive substance that serves as the vehicle or medium for a drug or other active substance. The New Oxford American Dictionary (2001). Excipients are inert substances which are commonly used as a diluent, vehicle, preservatives, binders, or stabilizing agent for drugs and includes, but not limited to, proteins (e.g., serum albumin, etc.), amino acids (e.g., aspartic acid, glutamic acid, lysine, arginine, glycine, histidine, etc.), fatty acids and phospholipids (e.g., alkyl sulfonates, caprylate, etc.), surfactants (e.g., SDS, polysorbate, nonionic surfactant, etc.), saccharides (e.g., sucrose, maltose, trehalose, etc.) and polyols (e.g., mannitol, sorbitol, etc.). See also Joseph P. Remington, Remington's Pharmaceutical Sciences, 18th ed., Mack Publishing Co., Easton, Pa.), which is hereby incorporated in its entirety.

As used herein, the term “—(C₁-C₁₀)alkyl” refers to a saturated straight chain or branched non-cyclic hydrocarbon having from 1 to 10 carbon atoms. Representative saturated straight chain —(C₁-C₁₀)alkyls include -methyl, -ethyl, -n-propyl, -n-butyl, -n-pentyl, -n-hexyl, -n-heptyl, -n-octyl, -n-nonyl, and -n-decyl. Representative saturated branched —(C₁-C₁₀)alkyls include -isopropyl, -sec-butyl, -isobutyl, -tert-butyl, -isopentyl, -2-methylbutyl, -3-methylbutyl, -2,2-dim ethylbutyl, -2,3-dimethylbutyl, -2-methylpentyl, -3-methylpentyl, -4-methylpentyl, -2-methylhexyl, -3-methylhexyl, -4-methylhexyl, -5-methylhexyl, -2,3-dimethylbutyl, -2,3-dimethylpentyl, -2,4-dimethylpentyl, -2,3-dimethylhexyl, -2,4-dimethylhexyl, -2,5-dimethylhexyl, -2,2-dimethylpentyl, -2,2-dimethylhexyl, -3,3-dimethylpentyl, -3,3-dimethylhexyl, -4,4-dimethylhexyl, -2-ethylpentyl, -3-ethylpentyl, -2-ethylhexyl, -3-ethylhexyl, -4-ethylhexyl, -2-methyl-2-ethylpentyl, -2-methyl-3-ethylpentyl, -2-methyl-4-ethylpentyl, -2-methyl-2-ethylhexyl, -2-methyl-3-ethylhexyl, -2-methyl-4-ethylhexyl, -2,2-diethylpentyl, -3,3-diethylhexyl, -2,2-diethylhexyl, -3,3-diethylhexyl and the like.

As used herein, the term “—(C₂-C₁₀)alkenyl” refers to a straight chain or branched non-cyclic hydrocarbon having from 2 to 10 carbon atoms and including at least one carbon-carbon double bond. Representative straight chain and branched —(C₂-C₁₀)alkyl-pentenyl, -2-pentenyl, -3-methyl-1-butenyl, -2-methyl-2-butenyl, -2,3-dimethyl-2-butenyl, -1-hexenyl, -2-hexenyl, -3-hexenyl, -1-heptenyl, -2-heptenyl, -3-heptenyl, -1-octenyl, -2-octenyl, -3-octenyl, -1-nonenyl, -2-nonenyl, -3-nonenyl, -1-decenyl, -2-decenyl, -3-decenyl and the like.

As used herein, the term “—(C₂-C₁₀)alkynyl” refers to a straight chain or branched non-cyclic hydrocarbon having from 2 to 10 carbon atoms and including at least one carbon-carbon triple bond. Representative straight chain and branched —(C₂-C₁₀)alkynyls include -acetylenyl, -propynyl, -1-butynyl, -2-butynyl, -1-pentynyl, -2-pentynyl, -3-methyl-1-butynyl, -4-pentynyl, -1-hexynyl, -2-hexynyl, -5-hexynyl, -1-heptynyl, -2-heptynyl, -6-heptynyl, -1-octynyl, -2-octynyl, -7-octynyl, -1-nonynyl, -2-nonynyl, -8-nonynyl, -1-decynyl, -2-decynyl, -9-decynyl and the like.

As used herein, the term “—(C₃-C₁₀)cycloalkyl” refers to a saturated cyclic hydrocarbon having from 3 to 10 carbon atoms. Representative (C₃-C₁₀)cycloalkyls include -cyclopropyl, -cyclobutyl, -cyclopentyl, -cyclohexyl, -cycloheptyl, -cyclooctyl, -cyclononyl and -cyclodecyl.

As used herein, the term “—(C₈-C₁₄)bicycloalkyl” refers to a bi-cyclic hydrocarbon ring system having from 8 to 14 carbon atoms and at least one saturated cyclic alkyl ring. Representative —(C₈-C₁₄)bicycloalkyls include -indanyl, -1,2,3,4-tetrahydronaphthyl, -5,6,7,8-tetrahydronaphthyl, -perhydronaphthyl and the like.

As used herein, the term “—(C₈-C₁₄)tricycloalkyl” refers to a tri-cyclic hydrocarbon ring system having from 8 to 14 carbon atoms and at least one saturated cycloalkyl ring. Representative —(C₈-C₁₄)tricycloalkyls include -pyrenyl, -1,2,3,4-tetrahydroanthracenyl, -perhydroanthracenyl, -aceanthreneyl, -1,2,3,4-tetrahydropenanthrenyl, -5,6,7,8-tetrahydrophenanthrenyl, -perhydrophenanthrenyl and the like.

As used herein, the term “—(C₄-C₁₀)cycloalkenyl” refers to a cyclic non-aromatic hydrocarbon having at least one carbon-carbon double bond in the cyclic system and from 4 to 10 carbon atoms. Representative (C₄-C₁₀)cycloalkenyls include -cyclobutenyl, -cyclopentenyl, -cyclopentadienyl, -cyclohexenyl, -cyclohexadienyl, -cycloheptenyl, -cycloheptadienyl, -cycloheptatrienyl, -cyclooctenyl, -cyclooctadienyl, -cyclooctatrienyl, -cyclooctatetraenyl, -cyclononenyl, -cyclononadienyl, -cyclodecenyl, -cyclodecadienyl and the like.

As used herein, the term “—(C₈-C₁₄)bicycloalkenyl” refers to a bi-cyclic hydrocarbon ring system having at least one carbon-carbon double bond in each ring and from 8 to 14 carbon atoms. Representative —(C₈-C₁₄)bicycloalkenyls include -indenyl, -pentalenyl, -naphthalenyl, -azulenyl, -heptalenyl, -1,2,7,8-tetrahydronaphthalenyl and the like.

As used herein, the term “—(C₈-C₁₄)tricycloalkenyl” refers to a tri-cyclic hydrocarbon ring system having at least one carbon-carbon double bond in each ring and from 8 to 14 carbon atoms. Representative —(C₈-C₁₄)tricycloalkenyls include -anthracenyl, -phenanthrenyl, -phenalenyl, -acenaphthalenyl, -asindacenyl, -s-indacenyl and the like.

As used herein, the term “-aryl” refers to an aromatic moiety from six to ten carbon atoms such as phenyl and naphthyl.

As used herein, the term “-substituted aryl” refers to an aryl moiety independently substituted with one to five groups selected from C₁₋₁₀ alkanoyloxy, hydroxyl, halo, C₁₋₁₀alkylamino, di-C₁₋₁₀ alkylamino, and amido.

As used herein, the term “-(3- to 6-membered) heterocycle” or “-(3- to 6-membered) heterocyclo” means a 3- to 6-membered monocyclic heterocyclic ring which is either saturated, unsaturated, non-aromatic or aromatic. A 3- or 4-membered heterocycle can contain up to 3 heteroatoms and a 5- or 6-membered heterocycle can contain up to 5 heteroatoms. Each heteroatom is independently selected from nitrogen, which can be quaternized; oxygen; and sulfur, including sulfoxide and sulfone. The -(3- to 6-membered)heterocycle can be attached via any heteroatom or carbon atom. Representative -(3- to 6-membered)heterocycles include furyl, thiophenyl, pyrrolyl, oxazolyl, imidazolyl, thiazolyl, isoxazolyl, pyrazolyl, isothiazolyl, triazinyl, pyrrolidinonyl, pyrrolidinyl, hydantoinyl, oxiranyl, oxetanyl, tetrahydrofuranyl, tetrahydrothiophenyl, pyridyl, pyrimidyl, pyridazinyl and the like.

As used herein, the term “—CH(halo)₂” refers to a methyl group wherein two of the hydrogens of the methyl group have been replaced with a halogen. Representative —CH(halo)₂ groups include —CHF₂, —CHCl₂, —CHBr₂, —CHBrCl, and —CHClI.

As used herein, the term “—C(halo)₃” refers to a methyl group wherein each of the hydrogens of the methyl group has been replaced with a halogen. Representative —C(halo)₃ groups include —CF₃, —CF₂Cl, —CCl₃, —CBr₃, —CFBr₂ and —Cl₃.

As used herein, the term “counterion” refers to a positively-charged moiety and includes ammonium and the cations of alkali metals such as sodium, potassium and lithium; alkaline earth metals such as calcium and magnesium; other metals, such as aluminum and zinc; organic amines, such as unsubstituted or hydroxy-substituted mono-, di- or trialkylamines; dicyclohexylamine; tributyl amine; pyridine; N-methyl-N-ethylamine; diethylamine; triethylamine; mono-, bis- or tris-(2-hydroxy-lower alkyl amines), such as mono-, bis- or tris-(2-hydroxyethyl)amine, 2-hydroxy-tert-butylamine or tris-(hydroxymethyl)methylamine; N,N-di-lower alkyl-N-(hydroxy lower alkyl)-amines, such as N,N-dimethyl-N-(2-hydroxyethyl)amine or tri-(2-hydroxyethyl)amine; N-methyl-D-glucamine; and amino acids such as arginine, lysine and the like.

As used herein, the term “halo” includes F, Cl, Br and I.

As used herein, the phrase “pharmaceutically acceptable salt” is a salt formed from an acid and a basic group of one of the compounds of Formula (I) [the configuration of C₁ can be either S (Sinister) or R (Rectus)]. Illustrative salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. The term “pharmaceutically acceptable salt” also refers to a salt prepared from a compound of Formula (I) having an acidic functional group, such as a carboxylic acid functional group, and a pharmaceutically acceptable inorganic or organic base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia and organic amines, such as unsubstituted or hydroxy-substituted mono-, di- or trialkylamines; dicyclohexylamine; tributyl amine; pyridine; N-methyl-N-ethylamine; diethylamine; triethylamine; mono-, bis- or tris-(2-hydroxy-lower alkyl amines), such as mono-, bis- or tris-(2-hydroxyethyl)amine, 2-hydroxy-tert-butylamine or tris-(hydroxymethyl)methylamine, N,N-di-lower alkyl-N-(hydroxy lower alkyl)-amines, such as N,N-dimethyl-N-(2-hydroxyethyl)amine or tri-(2-hydroxyethyl)amine; N-methyl-D-glucamine; and amino acids such as arginine, lysine and the like.

In general, the present invention comprises methods and compositions for the treatment of HCV and HBV infections, in vivo and in vitro. The invention comprises a method for inhibiting the infection or replication of HCV, comprising administering an effective amount of a composition comprising at least one of lamiridosin, a derivative of lamiridosin, or an iridoid or a combination thereof. The compositions comprise lamiridosin,

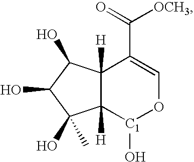

or a derivative of lamiridosin wherein the compound is

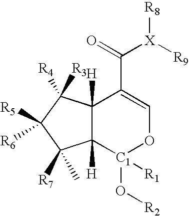

wherein R₁ is hydrogen, or R and OR₁ taken together can form an oxo group;
R₂ is hydrogen, β-D-glucopyranosyl group, (C₁₋₁₀)alkyl, (C₂₋₁₀)alkenyl, (C₂₋₁₀)alkynyl, or any of which groups can be optionally substituted with one to twenty-two of the same or different halogen atoms;
R₃ and R₄ are independently hydrogen, —OR′, —OC(O)R″, halo, R₃ and R₄ together can form an oxo group, and R′ and R″ are as described as below;
R₅ and R₆ are independently hydrogen, —OR′, —OC(O)R″, halo, R₅ and R₆ together can form an oxo group, and R′ and R″ are as described as below;
R₇ is hydrogen or hydroxy;
R₈ and R₉ are independently hydrogen, (C₁₋₁₀)alkyl, (C₂₋₁₀)alkenyl, (C₂₋₁₀)alkynyl, or any of which groups can be optionally substituted with one to twenty-two of the same or different halogen atoms;
R′ is hydrogen, (C₁₋₁₀)alkyl, (C₂₋₁₀)alkenyl, (C₂₋₁₀)alkynyl, or any of which groups can be optionally substituted with one to twenty-two of the same or different halogen atoms;
R″ is hydrogen, (C₁₋₁₀)alkyl, (C₂₋₁₀)alkenyl, (C₂₋₁₀)alkynyl, (C₃₋₁₀)cycloalkyl, (C₈₋₁₄)bicycloalkyl, (C₈₋₁₄)tricycloalkyl, (C₄₋₁₀)cycloalkenyl, (C₈₋₁₄)bicycloalkenyl, (C₈₋₁₄)tricycloalkenyl, aryl, substituted aryl, (3- to 6-membered)heterocycle, or any of which groups can be optionally substituted with one to twenty-two of the same or different halogen atoms; and
X is oxygen or nitrogen, or the compound is a derivative having the structure

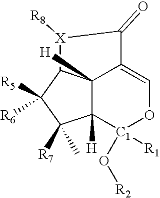

wherein R₁ is hydrogen, or R and OR₁ taken together can form an oxo group;
R₂ is hydrogen, β-D-glucopyranosyl group, (C₁₋₁₀)alkyl, (C₂₋₁₀)alkenyl, (C₂₋₁₀)alkynyl, or any of which groups can be optionally substituted with one to twenty-two of the same or different halogen atoms;
R₅ and R₆ are independently hydrogen, —OR′, —OC(O)R″, halo, R₅ and R₆ together can form an oxo group, and R′ and R″ are as described as below;
R₈ is hydrogen, (C₁₋₁₀)alkyl, (C₂₋₁₀)alkenyl, (C₂₋₁₀)alkynyl, or any of which groups can be optionally substituted with one to twenty-two of the same or different halogen atoms;
X is oxygen or nitrogen;
R′ is hydrogen, (C₁₋₁₀)alkyl, (C₂₋₁₀)alkenyl, (C₂₋₁₀)alkynyl, or any of which groups can be optionally substituted with one to twenty-two of the same or different halogen atoms;
R″ is hydrogen, (C₁₋₁₀)alkyl, (C₂₋₁₀)alkenyl, (C₂₋₁₀)alkynyl, (C₃₋₁₀)cycloalkyl, (C₈₋₁₄)bicycloalkyl, (C₈₋₁₄)tricycloalkyl, (C₄₋₁₀)cycloalkenyl, (C₈₋₁₄)bicycloalkenyl, (C₈₋₁₄)tricycloalkenyl, aryl, substituted aryl, (3- to 6-membered)heterocycle, or any of which groups can be optionally substituted with one to twenty-two of the same or different halogen atoms. The compositions comprise at least one iridoid, wherein the iridoid is aucubin, catapol, geniposide, loganinic acid, loganin, cornin, shanziside methyl ester, gardenoside, harpogoside, agnuside, picroside II, ammarogentin, sweroside, swertiamarin, oleuropein, aucubin aglycone, catapol aglycone, geniposide aglycone, loganinic acid aglycone, loganin aglycone, cornin aglycone, shanziside methyl ester aglycone, gardenoside aglycone, harpogoside aglycone, agnuside aglycone, picroside II aglycone, sweroside aglycone, swertiamarin aglycone, or oleuropein aglycone. The composition further comprises a pharmaceutically acceptable formulation, and further comprises at least one pharmaceutically active agent.

The invention comprises methods for inhibiting the infection or replication of HBV, comprising administering an effective amount of a composition comprising at least one of lamiridosin, a derivative of lamiridosin, or an iridoid or a combination thereof. A composition comprises lamiridosin,

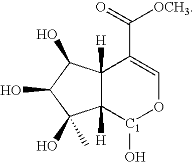

A composition comprises a derivative of lamiridosin, wherein the structure of derivative of lamiridosin is

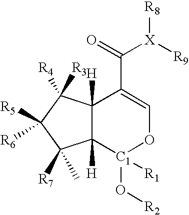

wherein R₁ is hydrogen, or R and OR₁ taken together can form an oxo group;
R₂ is hydrogen, β-D-glucopyranosyl group, (C₁₋₁₀)alkyl, (C₂₋₁₀)alkenyl, (C₂₋₁₀)alkynyl, or any of which groups can be optionally substituted with one to twenty-two of the same or different halogen atoms;
R₃ and R₄ are independently hydrogen, —OR′, —OC(O)R″, halo, R₃ and R₄ together can form an oxo group, and R′ and R″ are as described as below;
R₅ and R₆ are independently hydrogen, —OR′, —OC(O)R″, halo, R₅ and R₆ together can form an oxo group, and R′ and R″ are as described as below;
R₇ is hydrogen or hydroxy;
R₈ and R₉ are independently hydrogen, (C₁₋₁₀)alkyl, (C₂₋₁₀)alkenyl, (C₂₋₁₀)alkynyl, or any of which groups can be optionally substituted with one to twenty-two of the same or different halogen atoms;
R′ is hydrogen, (C₁₋₁₀)alkyl, (C₂₋₁₀)alkenyl, (C₂₋₁₀)alkynyl, or any of which groups can be optionally substituted with one to twenty-two of the same or different halogen atoms;
R″ is hydrogen, (C₁₋₁₀)alkyl, (C₂₋₁₀)alkenyl, (C₂₋₁₀)alkynyl, (C₃₋₁₀)cycloalkyl, (C₈₋₁₄)bicycloalkyl, (C₈₋₁₄)tricycloalkyl, (C₄₋₁₀)cycloalkenyl, (C₈₋₁₄)bicycloalkenyl, (C₈₋₁₄)tricycloalkenyl, aryl, substituted aryl, (3- to 6-membered)heterocycle, or any of which groups can be optionally substituted with one to twenty-two of the same or different halogen atoms; and
X is oxygen or nitrogen, or the derivative is

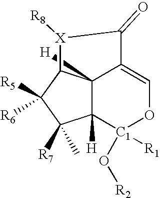

wherein R₁ is hydrogen, or R and OR₁ taken together can form an oxo group;
R₂ is hydrogen, β-D-glucopyranosyl group, (C₁₋₁₀)alkyl, (C₂₋₁₀)alkenyl, (C₂₋₁₀)alkynyl, or any of which groups can be optionally substituted with one to twenty-two of the same or different halogen atoms;
R₅ and R₆ are independently hydrogen, —OR′, —OC(O)R″, halo, R₅ and R₆ together can form an oxo group, and R′ and R″ are as described as below;
R₈ is hydrogen, (C₁₋₁₀)alkyl, (C₂₋₁₀)alkenyl, (C₂₋₁₀)alkynyl, or any of which groups can be optionally substituted with one to twenty-two of the same or different halogen atoms;
X is oxygen or nitrogen;
R′ is hydrogen, (C₁₋₁₀)alkyl, (C₂₋₁₀)alkenyl, (C₂₋₁₀)alkynyl, or any of which groups can be optionally substituted with one to twenty-two of the same or different halogen atoms;
R″ is hydrogen, (C₁₋₁₀)alkyl, (C₂₋₁₀)alkenyl, (C₂₋₁₀)alkynyl, (C₃₋₁₀)cycloalkyl, (C₈₋₁₄)bicycloalkyl, (C₈₋₁₄)tricycloalkyl, (C₄₋₁₀)cycloalkenyl, (C₈₋₁₄)bicycloalkenyl, (C₈₋₁₄)tricycloalkenyl, aryl, substituted aryl, (3- to 6-membered)heterocycle, or any of which groups can be optionally substituted with one to twenty-two of the same or different halogen atoms. A composition comprises at least one iridoid, wherein the iridoid is aucubin, catapol, geniposide, loganinic acid, loganin, cornin, shanziside methyl ester, gardenoside, harpogoside, agnuside, picroside II, ammarogentin, sweroside, swertiamarin, oleuropein, aucubin aglycone, catapol aglycone, geniposide aglycone, loganinic acid aglycone, loganin aglycone, cornin aglycone, shanziside methyl ester aglycone, gardenoside aglycone, harpogoside aglycone, agnuside aglycone, picroside II aglycone, sweroside aglycone, swertiamarin aglycone, or oleuropein aglycone. A composition further comprises at least one pharmaceutically active agent, or further comprises a pharmaceutically active agent.

The invention is directed to novel compounds comprising derivatives of lamiridosin, having the structure

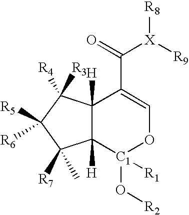

wherein R₁ is hydrogen, or R and OR₁ taken together can form an oxo group;
R₂ is hydrogen, β-D-glucopyranosyl group, (C₁₋₁₀)alkyl, (C₂₋₁₀)alkenyl, (C₂-10)alkynyl, or any of which groups can be optionally substituted with one to twenty-two of the same or different halogen atoms;
R₃ and R₄ are independently hydrogen, —OR′, —OC(O)R″, halo, R₃ and R₄ together can form an oxo group, and R′ and R″ are as described as below;
R₅ and R₆ are independently hydrogen, —OR′, —OC(O)R″, halo, R₅ and R₆ together can form an oxo group, and R′ and R″ are as described as below;
R₇ is hydrogen or hydroxy;
R₈ and R₉ are independently hydrogen, (C₁₋₁₀)alkyl, (C₂₋₁₀)alkenyl, (C₂₋₁₀)alkynyl, or any of which groups can be optionally substituted with one to twenty-two of the same or different halogen atoms;
R′ is hydrogen, (C₁₋₁₀)alkyl, (C₂₋₁₀)alkenyl, (C₂₋₁₀)alkynyl, or any of which groups can be optionally substituted with one to twenty-two of the same or different halogen atoms;
R″ is hydrogen, (C₁₋₁₀)alkyl, (C₂₋₁₀)alkenyl, (C₂₋₁₀)alkynyl, (C₃₋₁₀)cycloalkyl, (C₈₋₁₄)bicycloalkyl, (C₈₋₁₄)tricycloalkyl, (C₄₋₁₀)cycloalkenyl, (C₈₋₁₄)bicycloalkenyl, (C₈₋₁₄)tricycloalkenyl, aryl, substituted aryl, (3- to 6-membered)heterocycle, or any of which groups can be optionally substituted with one to twenty-two of the same or different halogen atoms; and
X is oxygen or nitrogen or the structure is

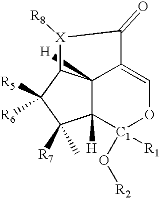

wherein R₁ is hydrogen, or R and OR₁ taken together can form an oxo group;
R₂ is hydrogen, β-D-glucopyranosyl group, (C₁₋₁₀)alkyl, (C₂₋₁₀)alkenyl, (C₂₋₁₀)alkynyl, or any of which groups can be optionally substituted with one to twenty-two of the same or different halogen atoms;
R₅ and R₆ are independently hydrogen, —OR′, —OC(O)R″, halo, R₅ and R₆ together can form an oxo group, and R′ and R″ are as described as below;
R₈ is hydrogen, (C₁₋₁₀)alkyl, (C₂₋₁₀)alkenyl, (C₂₋₁₀)alkynyl, or any of which groups can be optionally substituted with one to twenty-two of the same or different halogen atoms;
X is oxygen or nitrogen;
R′ is hydrogen, (C₁₋₁₀)alkyl, (C₂₋₁₀)alkenyl, (C₂₋₁₀)alkynyl, or any of which groups can be optionally substituted with one to twenty-two of the same or different halogen atoms;
R″ is hydrogen, (C₁₋₁₀)alkyl, (C₂₋₁₀)alkenyl, (C₂₋₁₀)alkynyl, (C₃₋₁₀)cycloalkyl, (C₈₋₁₄)bicycloalkyl, (C₈₋₁₄)tricycloalkyl, (C₄₋₁₀)cycloalkenyl, (C₈₋₁₄)bicycloalkenyl, (C₈₋₁₄)tricycloalkenyl, aryl, substituted aryl, (3- to 6-membered)heterocycle, or any of which groups can be optionally substituted with one to twenty-two of the same or different halogen atoms.

It must be noted that, as used in this specification and the appended claims, the singular forms “a,” “an,” and “the” include plural referents unless the context clearly dictates otherwise.

All patents, patent applications and references included herein are specifically incorporated by reference in their entireties.

It should be understood, of course, that the foregoing relates only to preferred embodiments of the present invention and that numerous modifications or alterations may be made therein without departing from the spirit and the scope of the invention as set forth in this disclosure.

The present invention is further illustrated by the following examples, which are not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims.

TABLE 5
DERIVATIVE COMPOUNDS

| NO. | COMPOUND |
|---|---|
| 1 | 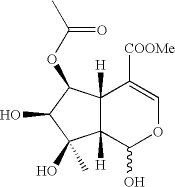 |
| 2 | 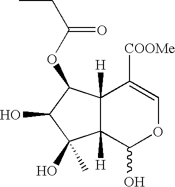 |
| 3 | 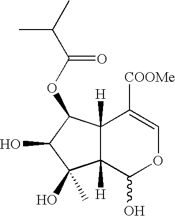 |
| 4 | 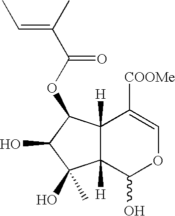 |
| 5 | 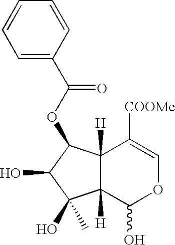 |
| 6 | 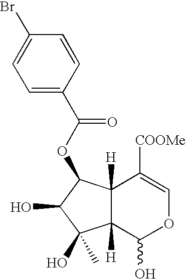 |
| 7 | 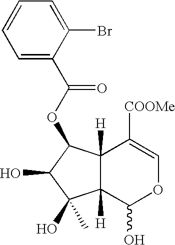 |
| 8 | 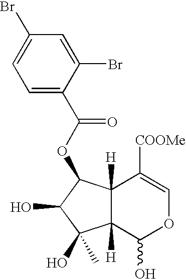 |
| 9 | 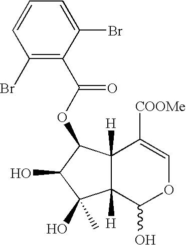 |
| 10 | 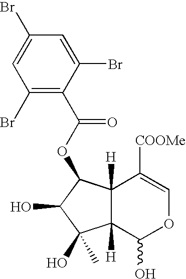 |
| 11 | 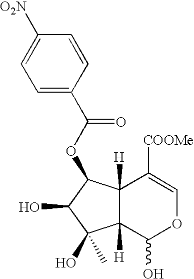 |
| 12 | 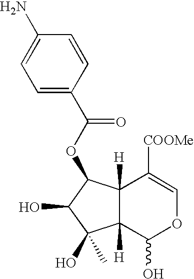 |
| 13 | 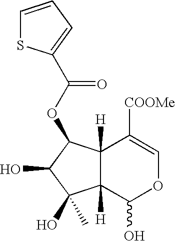 |
| 14 | 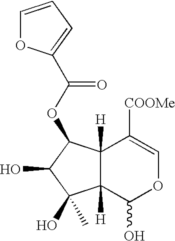 |
| 15 | 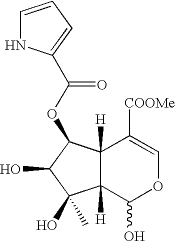 |
| 16 | 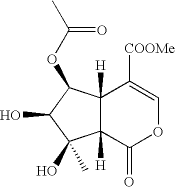 |
| 17 | 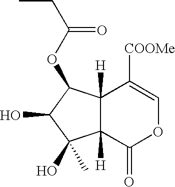 |
| 18 | 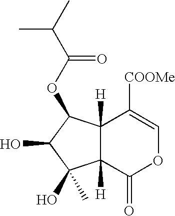 |
| 19 | 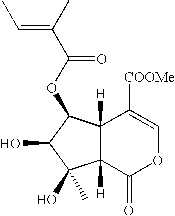 |
| 20 | 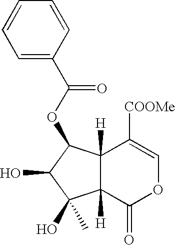 |
| 21 | 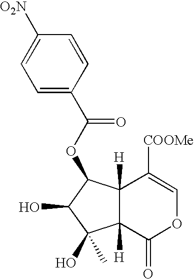 |
| 22 | 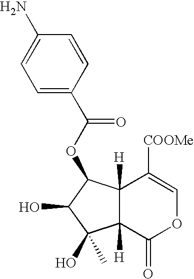 |
| 23 | 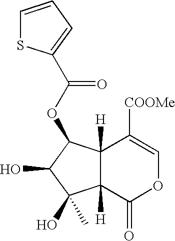 |
| 24 | 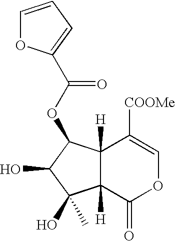 |
| 25 | 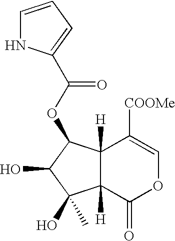 |
| 26 | 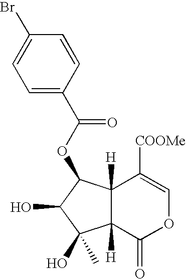 |
| 27 | 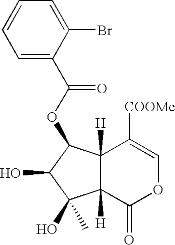 |
| 28 | 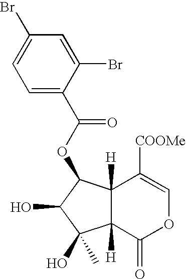 |
| 29 | 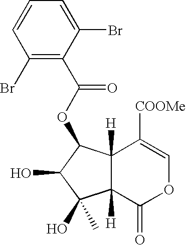 |
| 30 | 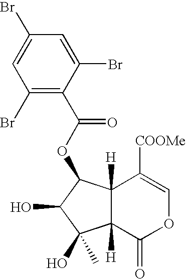 |
| 31 | 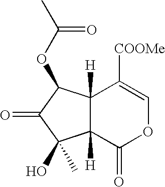 |
| 32 | 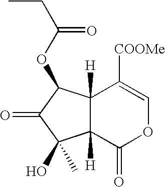 |
| 33 | 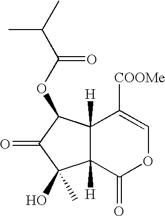 |
| 34 | 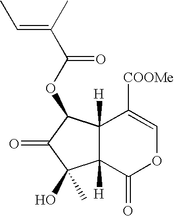 |
| 35 | 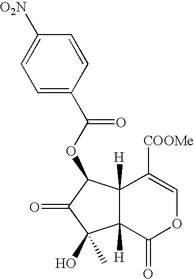 |
| 36 | 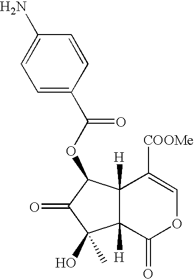 |
| 37 | 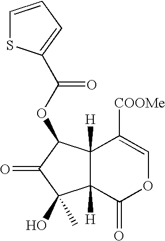 |
| 38 | 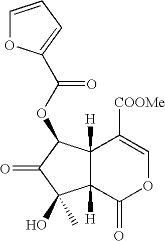 |
| 39 | 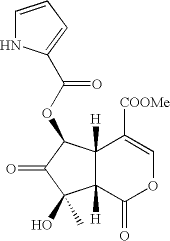 |
| 40 | 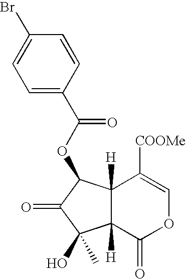 |
| 41 | 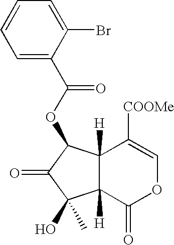 |
| 42 | 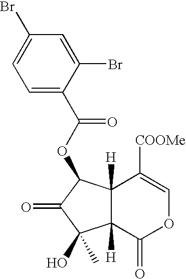 |
| 43 | 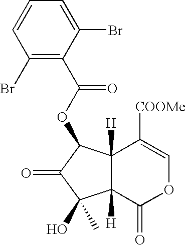 |
| 44 | 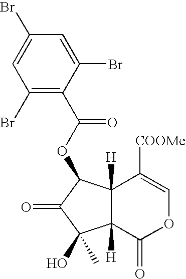 |
| 45 | 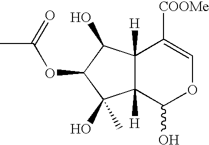 |
| 46 | 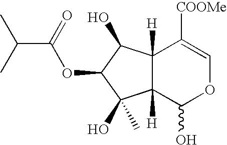 |
| 47 | 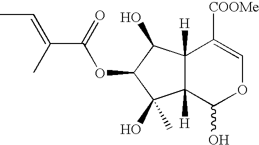 |
| 48 | 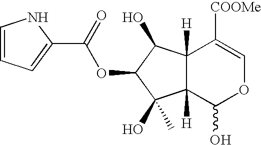 |
| 49 | 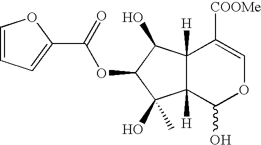 |
| 50 | 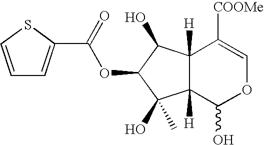 |
| 51 | 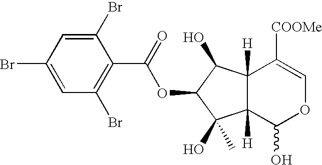 |
| 52 | 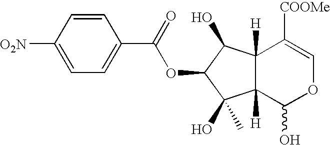 |
| 53 | 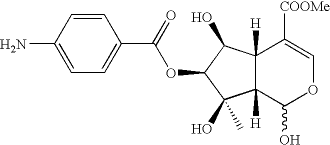 |
| 54 | 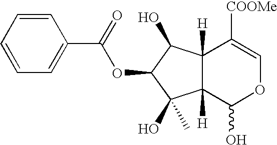 |
| 55 | 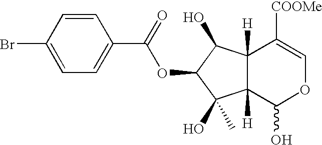 |
| 56 | 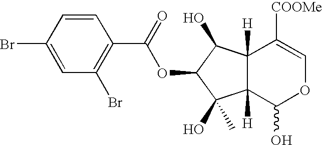 |
| 57 | 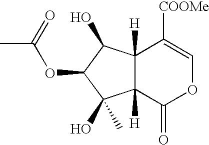 |
| 58 | 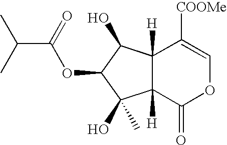 |
| 59 | 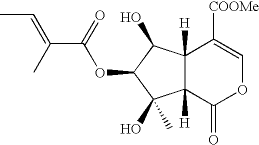 |
| 60 | 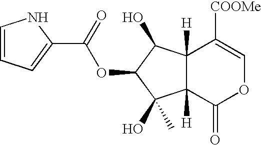 |
| 61 | 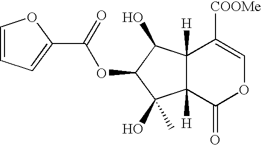 |
| 62 | 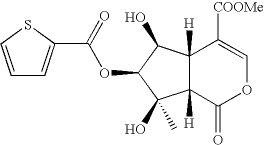 |
| 63 | 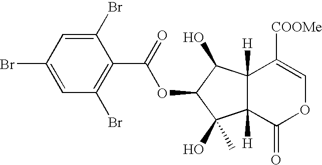 |
| 64 | 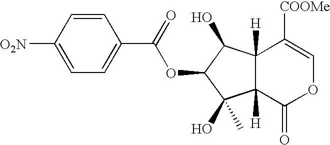 |
| 65 | 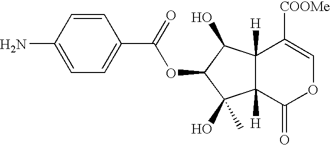 |
| 66 | 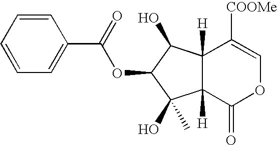 |
| 67 | 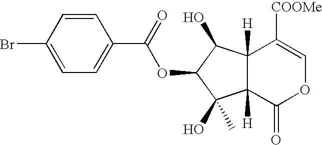 |
| 68 | 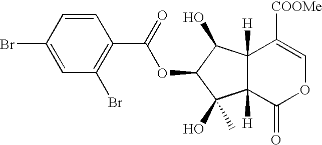 |
| 69 | 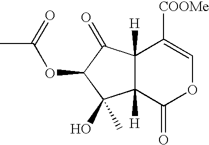 |
| 70 | 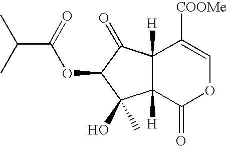 |
| 71 | 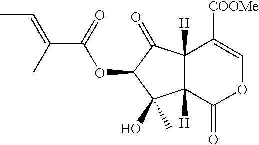 |
| 72 | 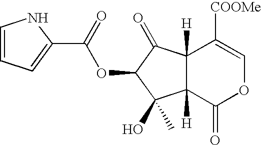 |
| 73 | 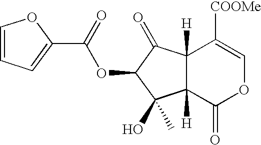 |
| 74 | 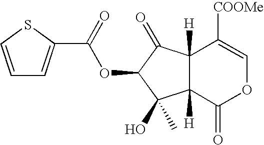 |
| 75 | 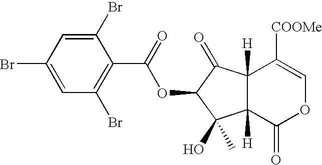 |
| 76 | 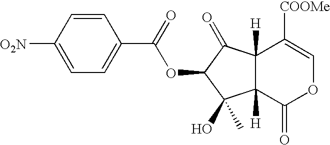 |
| 77 | 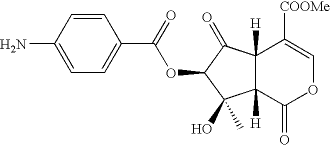 |
| 78 | 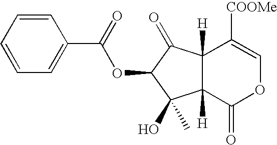 |
| 79 | 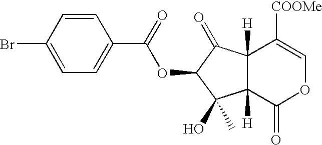 |
| 80 | 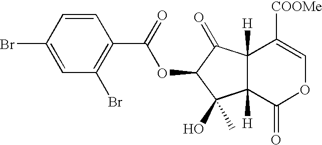 |
| 81 | 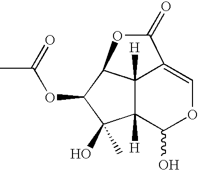 |
| 82 | 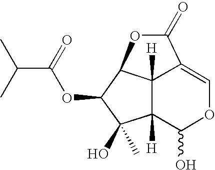 |
| 83 | 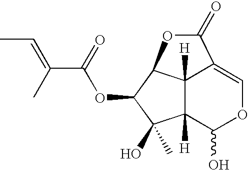 |
| 84 | 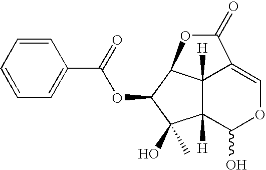 |
| 85 | 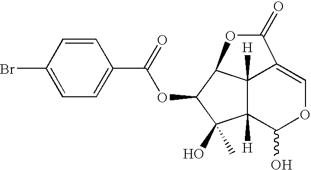 |
| 86 | 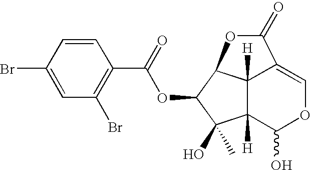 |
| 87 | 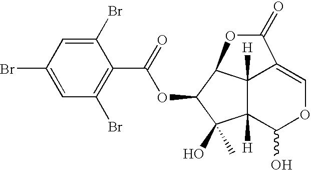 |
| 88 | 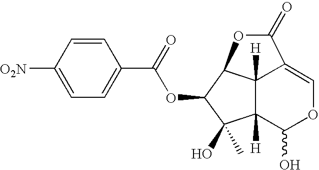 |
| 89 | 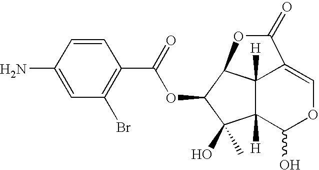 |
| 90 | 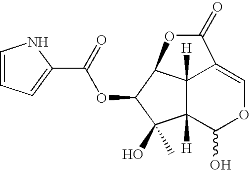 |
| 91 | 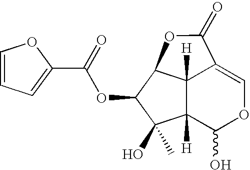 |
| 92 | 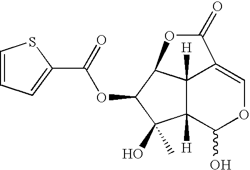 |
| 93 | 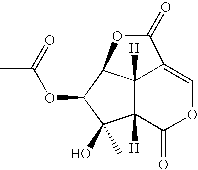 |
| 94 | 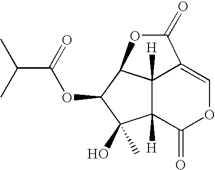 |
| 95 | 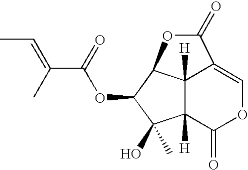 |
| 96 | 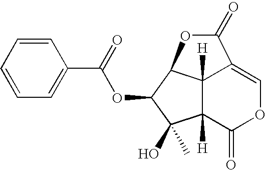 |
| 97 | 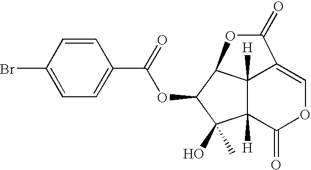 |
| 98 | 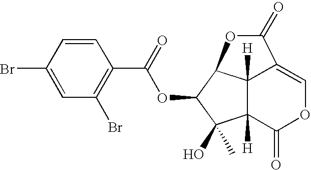 |
| 99 | 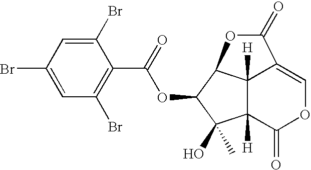 |
| 100 | 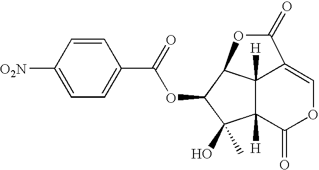 |
| 101 | 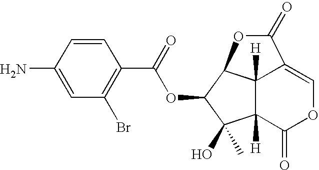 |
| 102 | 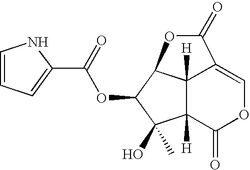 |
| 103 | 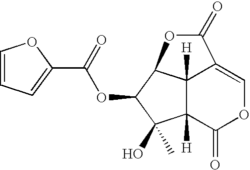 |
| 104 | 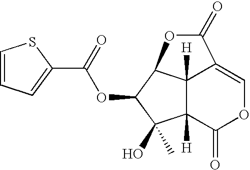 |
| 105 | 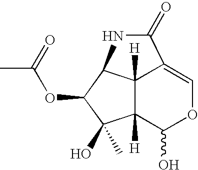 |
| 106 | 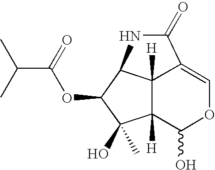 |
| 107 | 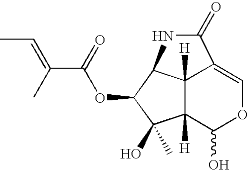 |
| 108 | 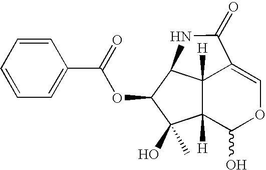 |
| 109 | 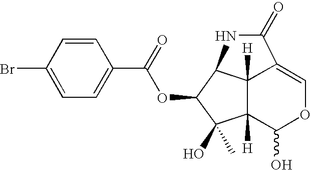 |
| 110 | 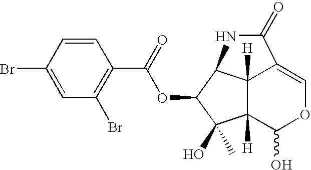 |
| 111 | 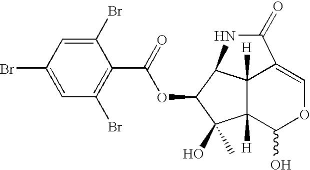 |
| 112 | 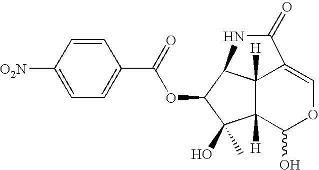 |
| 113 | 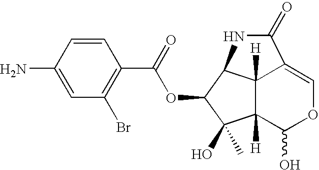 |
| 114 | 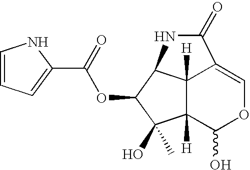 |
| 115 | 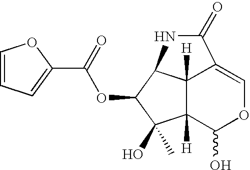 |
| 116 | 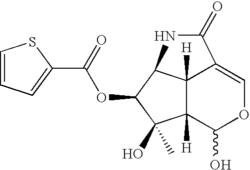 |
| 117 | 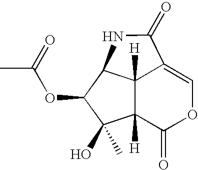 |
| 118 | 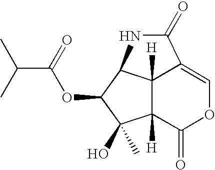 |
| 119 | 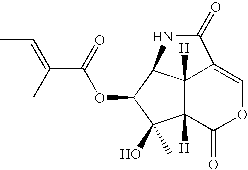 |
| 120 | 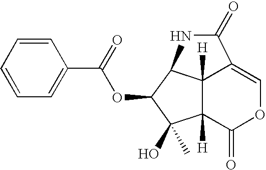 |
| 121 | 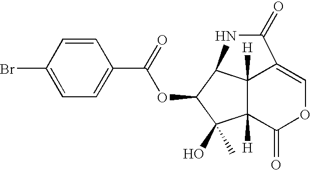 |
| 122 | 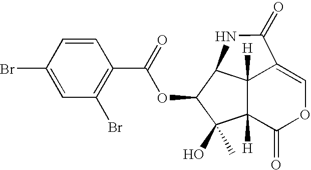 |
| 123 | 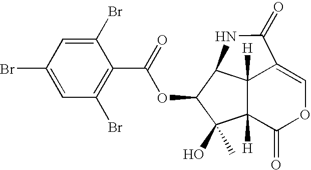 |
| 124 | 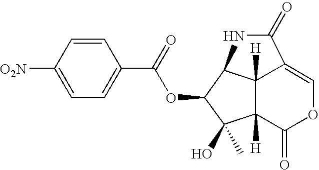 |
| 125 | 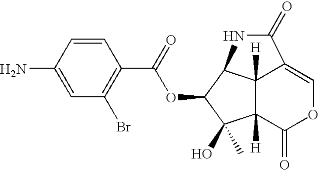 |
| 126 | 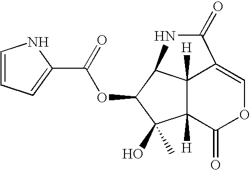 |
| 127 | 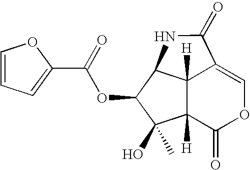 |
| 128 | 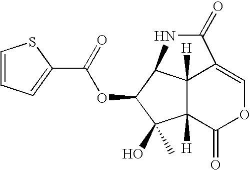 |

TABLE 7
IRIDOIDS

| | |
|---|---|
| 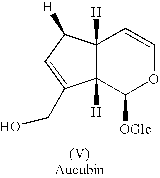 | 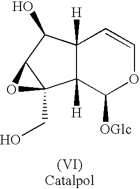 |
| 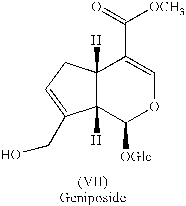 | 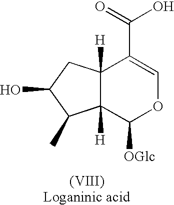 |
| 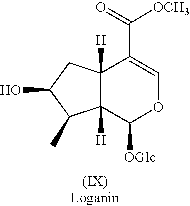 | 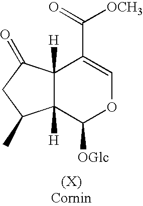 |
| 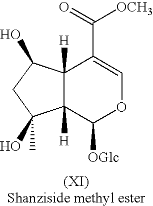 | 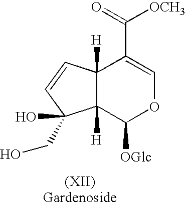 |

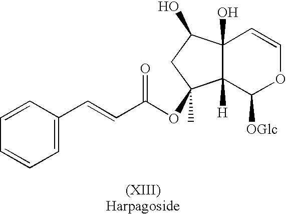

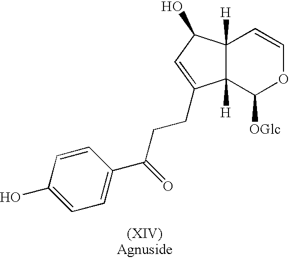

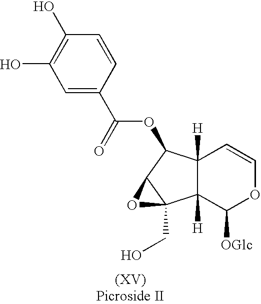

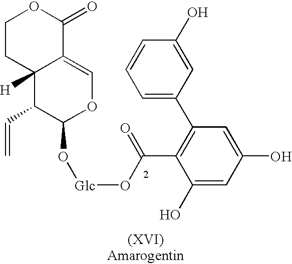

| | |
|---|---|
| 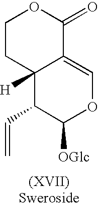 | 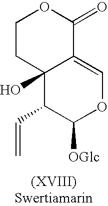 |

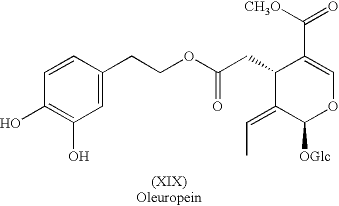

| | |
|---|---|
| 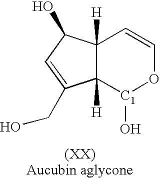 | 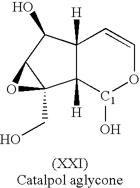 |
| 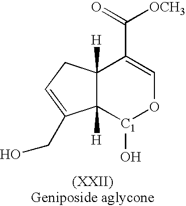 | 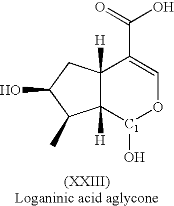 |
| 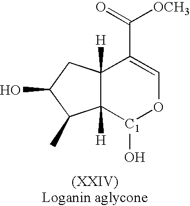 | 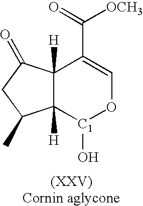 |
| 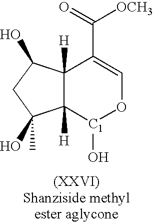 | 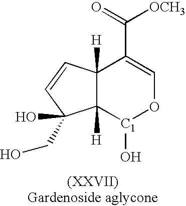 |

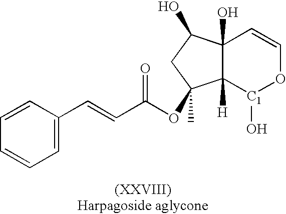

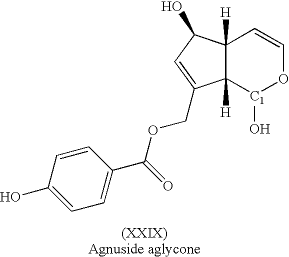

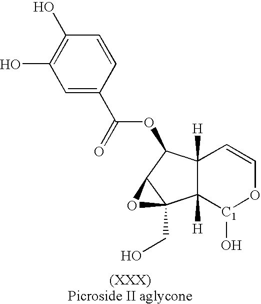

| | |
|---|---|
| 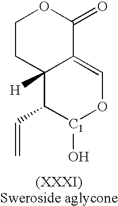 | 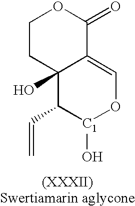 |

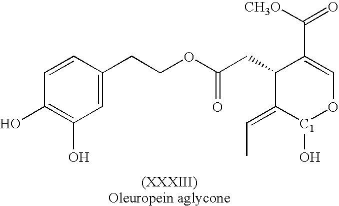

### EXAMPLES

### Example 1

### Assay and Protocols for Anti-HBSAg Assay (Procedure A)

The HBV-producing human hepatoblastoma cell line, Hep G2.2.15 was maintained in DMEM supplemented with 10% fetal bovine serum (FBS), 2 mM glutamine, 100 units/ml penicillin, 100 μg/ml streptomycin and 380 μg/ml gentamicin sulfate (G418) at 37° C. under 5% CO₂ in air. The Hep G2 parental cells were grown under identical conditions in MEM with Earle's salts without G418.

Hep G2.2.15 cells (190 μl) were seeded onto microtiter plates at a concentration of 20×10⁴ cells/ml. The cells incubated overnight in medium without G418 after which the compound compositions (10 μl) were added. Five 2-fold serial dilutions of extracts or fractions were tested starting at 200 μg/ml final culture concentration. PBS and 3TC (lamivudine) were included as the negative and positive controls, respectively. The treated cells were incubated at 37° C. for 4 days, after which the hepatitis B surface antigen (HBsAg) production in culture supernatants was assayed. The HBsAg released into the culture medium in the presence or absence of test samples was assayed using the BIO-RAD Genetic Systems HBsAg EIA 3.0 kit (Bio-Rad, Redmond, Wash.). This enzyme immunoassay is based on the capture of HBsAg on monoclonal antibody-coated plates. Four- and twelve-fold dilutions of all culture supernatants collected were assayed in triplicate wells of the EIA plate. The percentage inhibition of HBsAg secretion in the presence of the plant samples was calculated against the negative control (PBS).

The results of the activity in the assay for four compounds are shown in Table 1. All samples in Table 1 are 50 μg/ml

TABLE 1
Anti-HBSAg Activity (Procedure A)

| | % Control | | % Cell |
|---|---|---|---|
| Compound | HBSAg | % Inhibition | Viability |
| lamiridoside | 96 | 4 | 100 |
| lamiridosin | 100 | 0 | 100 |
| aucubin | 100 | 0 | 100 |
| aucubin enzyme | 96 | 4 | 100 |
| hydrolysate | | | |

### Example 2

### Anti-HBSAg Assay (Procedure B)

### Cell Culture

The HBV-producing human hepatoblastoma cell line, Hep G2 2.2.15 is maintained in DMEM supplemented with 10% fetal bovine serum (FBS), 2 mM glutamine, 100 units/ml penicillin, 100 μg/ml streptomycin at 37° C. under 5% CO₂ in air.

### Sample Preparation

Samples were dissolved in DMSO at the concentration of 20 mg/mL, then further diluted with DMEM medium to the appropriate concentration.

### Cytotoxicity Assay

Hep G2 2.2.15 cells (120 μl) were seeded onto 96-well plates at a concentration of 2×105 cells/mL. After incubation for 72 h, serial dilutions of the test samples were added to monolayer Hep G2 2.2.15 cells, and culture at 37° C. in a humidified atmosphere of 5% CO₂ in air for an additional 9 days, and the medium with sample was changed every 3 days, 10 μL 10% aqueous DMSO was used as control group. After incubation, the supernatant was taken out and 90 μL of DMEM and 10 μL of the 5 mg/ml MTT (3-[4,5-dimethylthiazol-2-yl]-2,5-diphenyltetrazolium bromide) solution was then added to each well, and the plates were incubated for an additional 3 hours. The MTT was removed, and to each well was added 100 μl of DMSO. The plates were agitated on an orbital shaker at 50 rpm at room temperature for 5 min. The optical density of each sample was measured at 490 nm. The percent absorbance of the samples treated with the test material was compared to the untreated sample to calculate the percentage of control. Data are expressed as relative viability, with cytotoxicity indicated by <100% relative viability.

### Assay of Antiviral Activity

Hep G2 2.2.15 cells (120 μl) were seeded onto 96-well plates at a concentration of 2×105 cells/mL. The cells incubated for 72 h in the medium without G418, after which the test samples (120 μl) were added. Five 3-fold serial dilutions of samples were tested. PBS and 3TC (lamivudine) were included as the negative and positive controls, respectively. The treated cells were incubated at 37° C. for 9 days; with the medium with sample being changed each 3 days, after which the culture supernatant was removed for analysis of both the hepatitis B surface antigen (HBsAg) production and hepatitis B DNA expression.

### Analysis of HBsAg

The HBsAg released into the culture medium in the presence or absence of test material was assayed using the kit manufactured by Shanghai Siic Kehua Biotech Co., Ltd (Shanghai, China). This enzyme immunoassay is based on the capture of HBsAg on monoclonal antibody-coated plates. Four- and twelve-fold dilutions of all culture supernatants collected were assayed in triplicate wells of the kit plate. The percentage inhibition of HBsAg secretion in the presence of the compound was calculated against the negative control (PBS). The IC50 values, the dose that inhibited viruses by 50%, were calculated using non-linear regression analysis (percent survival versus concentration).

TABLE 2
Anti-HBSAg Activity (Procedure B)

| | HBsAg | | | | | | HBeAg | | | | |
|---|---|---|---|---|---|---|---|---|---|---|---|
| Sample | μg/mL | OD1 | OD2 | OD3 | Ave. | Inhibition % | OD1 | OD2 | OD3 | Ave | Inhibition % |
| Lamiridoside | 200 | 1.546 | 1.504 | 1.531 | 1.527 | 0 | 1.095 | 1.634 | 1.621 | 1.450 | 0 |
| | 100 | 1.584 | 1.469 | 1.468 | 1.507 | 0 | 1.607 | 1.502 | 1.565 | 1.558 | 0 |
| | 50 | 1.659 | 1.504 | 1.255 | 1.473 | 0 | 1.774 | 1.669 | 1.482 | 1.642 | 0 |
| | 25 | 1.604 | 1.418 | 1.401 | 1.474 | 0 | 1.558 | 1.488 | 1.49 | 1.512 | 0 |
| | 12.5 | 1.305 | 1.149 | 1.331 | 1.262 | 0 | 1.509 | 1.254 | 1.345 | 1.369 | 0 |
| Lamiridosins | 150 | Toxic | / | / | / | / | / | / | / | / | / |
| A/B | 75 | Toxic | / | / | / | / | / | / | / | / | / |
| | 37.5 | 0.797 | 0.744 | 0.755 | 0.765 | 29.4 | 1.073 | 1.275 | 1.238 | 1.195 | 12.1 |
| | 18.75 | 1.208 | 1.105 | 1.107 | 1.140 | 0 | 1.151 | 1.171 | 1.410 | 1.244 | 8.5 |
| | 9.375 | 1.080 | 1.220 | 1.157 | 1.152 | 0 | 1.455 | 1.416 | 1.109 | 1.327 | 2.7 |
| Aucubin | 200 | 1.546 | 1.616 | 1.46 | 1.541 | 0 | 1.350 | 1.305 | 1.415 | 1.357 | 0 |
| (Reference | 100 | 1.276 | 1.353 | 1.246 | 1.292 | 0 | 1.707 | 1.479 | 1.662 | 1.616 | 0 |
| Control #1) | 50 | 1.218 | 1.375 | 1.222 | 1.272 | 0 | 1.357 | 1.437 | 1.354 | 1.383 | 0 |
| | 25 | 1.242 | 1.384 | 1.302 | 1.309 | 0 | 1.332 | 1.369 | 1.390 | 1.364 | 0 |
| | 12.5 | 1.196 | 1.261 | 1.238 | 1.232 | 0 | 1.397 | 1.419 | 1.318 | 1.378 | 0 |
| Negative | | 1.046 | 1.109 | 1.096 | 1.084 | | 1.229 | 1.496 | 1.354 | 1.360 | |
| Control | | | | | | | | | | | |
| Aucubin | 100 | 0.667 | 0.641 | 0.66 | 0.656 | 52.6 | 1.162 | 1.391 | 1.435 | 1.329 | 0 |
| aglycone | 50 | 0.935 | 0.926 | 0.675 | 0.845 | 37.9 | 1.295 | 1.259 | 1.123 | 1.226 | 0 |
| (Reference | 25 | 1.608 | 1.116 | 1.175 | 1.300 | 6.1 | 1.375 | 1.335 | 1.293 | 1.334 | 0 |
| Control #2) | 12.5 | 1.370 | 1.372 | 1.227 | 1.323 | 4.5 | 1.561 | 1.316 | 1.303 | 1.393 | 0 |
| | 6.25 | 1.392 | 1.375 | 1.454 | 1.407 | 0 | 1.327 | 1.265 | 1.304 | 1.299 | 0 |
| Negative | | 1.406 | 1.364 | 1.385 | 1.385 | | 1.192 | 1.317 | 1.156 | 1.222 | |
| Control | | | | | | | | | | | |

Aucubin aglycone (Reference Control #2) IC₅₀ = 70.79 μg/ml;

TABLE 3
Anti-HBV DNA Activity

| Sample | | | |
|---|---|---|---|
| (μg/ml) | copies/ml | Average | Inhibit |
| HP-S1 | 1.17E+06 | 8.40 × 10⁵ ± | 28.20% |
| 200 | 5.77E+05 | 3.02 × 10⁵ | |
| | 7.74E+05 | | |
| 100 | 1.50E+06 | 1.02 × 10⁶ ± | 12.80% |
| | 7.78E+05 | 4.13 × 10⁵ | |
| | 7.93E+05 | | |
| 50 | 1.26E+06 | 1.16 × 10⁶ ± | 0.85% |
| | 1.14E+06 | 9.61 × 10⁴ | |
| | 1.07E+06 | | |
| 25 | 1.95E+06 | 1.64 × 10⁶ ± | 0.00% |
| | 1.30E+06 | 3.26 × 10⁵ | |
| | 1.67E+06 | | |
| 12.5 | 1.32E+06 | 1.18 × 10⁶ ± | 0.00% |
| | 9.64E+05 | 1.91 × 10⁵ | |
| | 1.26E+06 | | |
| | 9.68E+04 | | |
| | 1.07E+05 | | |
| HP-S2 | 5.06E+05 | 6.49 × 10⁵ ± | 44.50% |
| 37.5 | 8.57E+05 | 1.84 × 10⁵ | |
| | 5.85E+05 | | |
| 18.75 | 8.22E+05 | 8.90 × 10⁵ ± | 23.90% |
| | 8.73E+05 | 7.84 × 10⁴ | |
| | 9.76E+05 | | |
| 9.37 | 1.12E+6 | 9.81 × 10⁵ ± | 0.00% |
| | 1.01E+06 | 2.55 × 10⁵ | |
| | 1.22E+06 | | |
| | 9.60E+05 | | |
| | 1.51E+06 | | |
| HP-S3 | 1.38E+06 | 1.20 × 10⁶ ± | 0.00% |
| 200 | 9.71E+05 | 2.08 × 10⁵ | |
| | 1.24E+06 | | |
| 100 | 1.17E+06 | 1.32 × 10⁶ ± | 0.00% |
| | 1.15E+06 | 2.83 × 10⁵ | |
| | 1.65E+06 | | |
| 50 | 1.22E+06 | 1.28 × 10⁶ ± | 0.00% |
| | 1.27E+06 | 6.56 × 10⁴ | |
| | 1.35E+06 | | |
| 25 | 9.52E+05 | 1.03 × 10⁵ ± | 1.35% |
| | 1.07E+06 | 1.76 × 10⁵ | |
| | 1.04E+06 | | |
| 12.5 | 1.03E+06 | 1.06 × 10⁶ ± | 9.40% |
| | 9.68E+05 | 1.09 × 10⁵ | |
| | 1.18E+06 | | |
| HP-S4 | 7.90E+05 | 8.43 × 10⁵ ± | 27.90% |
| 100 | 7.94E+05 | 8.78 × 10⁴ | |
| | 9.44E+05 | | |
| 50 | 1.05E+06 | 9.79 × 10⁵ ± | 16.30% |
| | 9.27E+05 | 6.38 × 10⁴ | |
| | 9.59E+05 | | |
| 25 | 1.02E+06 | 1.01 × 10⁶ ± | 13.70% |
| | 9.74E+05 | 3.38 × 10⁴ | |
| | 1.04E+06 | | |
| 12.5 | 1.16E+06 | 9.52 × 10⁵ ± | 8.60% |
| | 9.17E+05 | 1.93 × 10⁵ | |
| | 7.78E+05 | | |
| 6.25 | 1.26E+06 | 1.10 × 10⁶ ± | 5.99% |
| | 9.83E+05 | 1.43 × 10⁵ | |
| | 1.06E+06 | | |

Sample:
HP-S1 = Lamiridoside
HP-S2 = Lamiridosins A/B
HP-S3 = Aucubin (Reference Control #1)
HP-S4 = Aucubin aglycone (Reference Control #2)

Lamiridosin exerted a direct inhibition on the infectivity of the hepatitis C virus (Anti-HCV) ca. 85% at a dose of 100 μg/ml, see FIG. 1. The IC₅₀ of lamiridosin in the anti-HCV assay was found to be 0.6 μg/ml (2.31 μmol) (FIG. 2 and Table 4). The data in FIG. 2 were fit to a simple IC₅₀ binding isotherm. The resulting fitted parameters are shown in the plot. The result clearly indicates that lamiridosin A/B potently inhibits HCV viral entry with an IC₅₀ value of 0.6 ug/mL. This compound was non-cytotoxic to the Hep G2 2.2.15 cells at doses greater than 50 μg/ml.

TABLE 4

| [Sample 2] | Trial 1 | Exp2 |
|---|---|---|
| ug/mL | (% Inhib) | (% Inhib) |
| 1 | 70.1 | 56.6 |
| 5 | 81.2 | 68.4 |
| 10 | 86.8 | 81.8 |
| 50 | 96.1 | 96.7 |
| 100 | 99.5 | 99.5 |

### Example 3

### Analysis of Lamiridosin for Other Antiviral Activities

The aims of this example are to analyze the anti-viral spectrum of lamiridosin against other viruses.

### Experimental Design:

The four test substances was subjected to analysis for their ability to inhibit the entry (infectivity) of the influenza, Marburg, Ebola, and SARS virus entry assays at a dose of 100 μg/mL. The anti-HIV effect was tested separately in the HOG.R5 assay.

### Results:

These assays were completed and the results summarized in the FIG. 4. Lamiridosin (S2 in FIG. 4), at 50 mg·mL, is inactive against influenza, Marburg, SARS, Ebola, and HIV (inactive against the HOG.R5 assay at 20 mcg/ml).

### Example 4

### Anti-HBV DNA Assay

### Cell Culture

The HBV-producing human hepatoblastoma cell line, Hep G2 2.2.15 was maintained in DMEM supplemented with 10% fetal bovine serum (FBS), 2 mM glutamine, 100 units/ml penicillin, 100 μg/ml streptomycin at 37° C. under 5% CO2 in air.

### Sample Preparation

Samples were dissolved in DMSO at the concentration of 20 mg/mL, then further diluted with DMEM medium to the appropriate concentration.

### Cytotoxicity Assay

Hep G2 2.2.15 cells (120 μl) were seeded onto 96-well plates at a concentration of 2×105 cells/mL. After incubation for 72 h, serial dilutions of the test samples were added to monolayer Hep G2 2.2.15 cells, and culture at 37° C. in a humidified atmosphere of 5% CO₂ in air for an additional 9 days, and the medium with sample was changed every 3 days, 10 μL 10% aqueous DMSO was used as control group. After incubation, the supernatant was taken out and 90 μL of DMEM and 10 μL of the 5 mg/ml MTT (3-[4,5-dimethylthiazol-2-yl]-2,5-diphenyltetrazolium bromide) solution was then added to each well, and the plates were incubated for an additional 3 hours. The MTT was removed, and to each well was added 100 μl of DMSO. The plates were agitated on an orbital shaker at 50 rpm at room temperature for 5 min. The optical density of each sample was measured at 490 nm. The percent absorbance of the samples treated with the test material was compared to the untreated sample to calculate the percentage of control. Data are expressed as relative viability, with cytotoxicity indicated by <100% relative viability.

### Analysis of HBV DNA

DNA was extracted from 50 μL of the cultured supernatant using the QIAamp DNA Mini Kit (Qiagen, Hilden, Germany) according to the manufacturer's instructions. DNA was eluted into 50 μL nuclease-free water, and 2 μl added to a 50 μl PCR reaction mixture. The reaction was carried out using Taqman Probe reaction mix (ABI). The kit contained HotStarTaq polymerase which was included to avoid false positives in the quantitative PCR. The probe sequences were FAM-5′-TgT gTC TgC ggC gTT TTA TCA T -3′-TAMRA The primer sequences were 5′-TgT CCT ggT TAT CgC Tgg-3′ (sense) and 5′ CAA ACg ggC AAC ATA CCT T-3′ (antisense) designed to amplify a 115 base pair product from positions 292 to 407 of the HBV genome. Thermal cycling was performed in an IQ5 fluorescence quantify PCR instrument (Bio-Rad, America). Reaction conditions were: 95° C. for 15 minutes followed by 40 cycles of 94° C. for 20 seconds, 60° C. for 40 seconds. A five point standard curve (2.42×108 copies per milliliter (cpm), 2.59×107 cpm, 2.71×106 cpm, 3.05×105 cpm, 2.53×104 cpm) was generated from a high titre HBV serum. The calibration of this standard was confirmed by comparison with an International HBV DNA standard, (97/746) (NIBSC, Potters Bar, UK). Each test run included positive and negative controls.

### Anti-HVC Assay

The assay is utilized for showing the antiviral compounds that can prevent hepatitis-C(HCV) virus entry is a procedure based on an entry assay developed for the Ebola virus by Manicassamy et al. (2005).

293T producer cells were co-transfected with two vectors for production of virions that were used in Step 2. These virions were based on HIV and contain the viral protein of interest. For the assays disclosed herein, the protein utilized was the envelop protein for Hepatitis C. In Step 2, the virions were used to infect a host, e.g. HUH7 cells. If the host cell got infected, it produced luciferase and these cells will “light” up when the luciferase substrate was added. If a compound or mixture of compounds inhibited viral entry then the amount of luciferase produced will be significantly decreased.

The substances to be tested are first incubated with the HCV/HIV (or VSVG/HIV) pseudovirions, and the mixtures used to challenge the target cells (HUH7, a human liver-derived cell lines). Forty-eight hours post-infection, the infected cells were lysed and the luciferase level of the infected cells was determined following the protocol of Manicassamy et al. (2005).

See Tables 4 and 6 for selected compounds that showed inhibition in this assay.

TABLE 6

| | | Enzyme Hydrolysis | HCV |
|---|---|---|---|
| | | Processing | Inhibition |
| Code | Structure | Time (hours) | at 20 μg/mL |
| RD-4 | 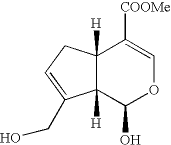 | No enzymeprocessing | 73.2 |
| RD-16RD-16 | 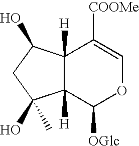 | 4.00.5 | 61.249.0 |
| RD-8RD-8 | 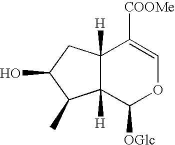 | 4.00.5 | 41.424.8 |
| RD-17RD-17 | 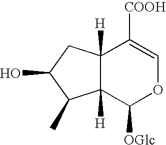 | 4.00.5 | 52.539.4 |
| RD-18RD-18 | 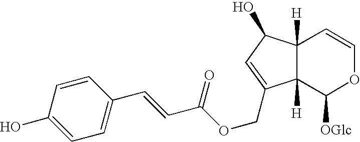 | 4.00.5 | 56.646.6 |

### Example 5

### HCV Viral (Live) Assay

Protocol for small molecule inhibition of the HCV JFH1 infectivity assay (JFH1 Foci Reduction Assay) This example showed which selected small organic molecules that were active in the HCV entry (HCVpp) (pseudotype) assay had activity to inhibit HCV JFH1 infectivity (HCVcc) (live virus) in-vitro. The inhibitory activity of the compounds was measured via a JFH1 foci reduction assay as compared to a non-treated JFH1 infection.

Huh 7 cells were plated and treated either by: (1) Cells were pre-incubated with compounds, rinsed, and then virus was added or (2) Virus was preincubated with the compound and then added to the Huh 7 cells (compound present during entire infection cycle). 48 hr post infection, cells were assayed for plaque formation. Assays were performed in triplicate.

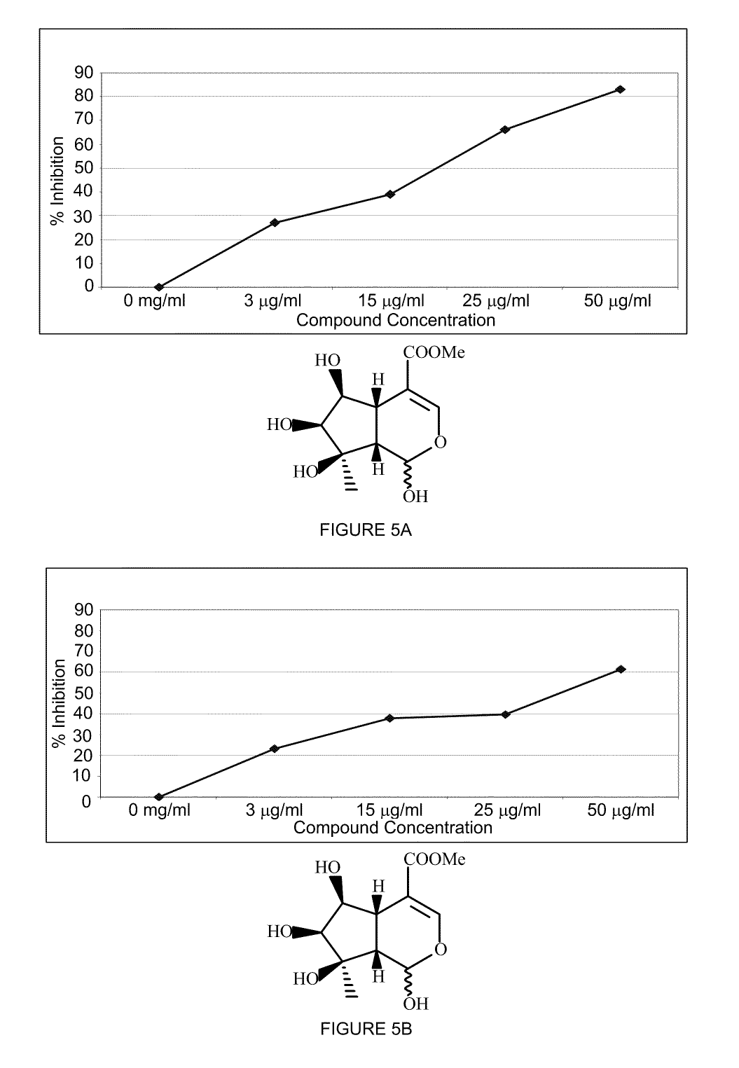

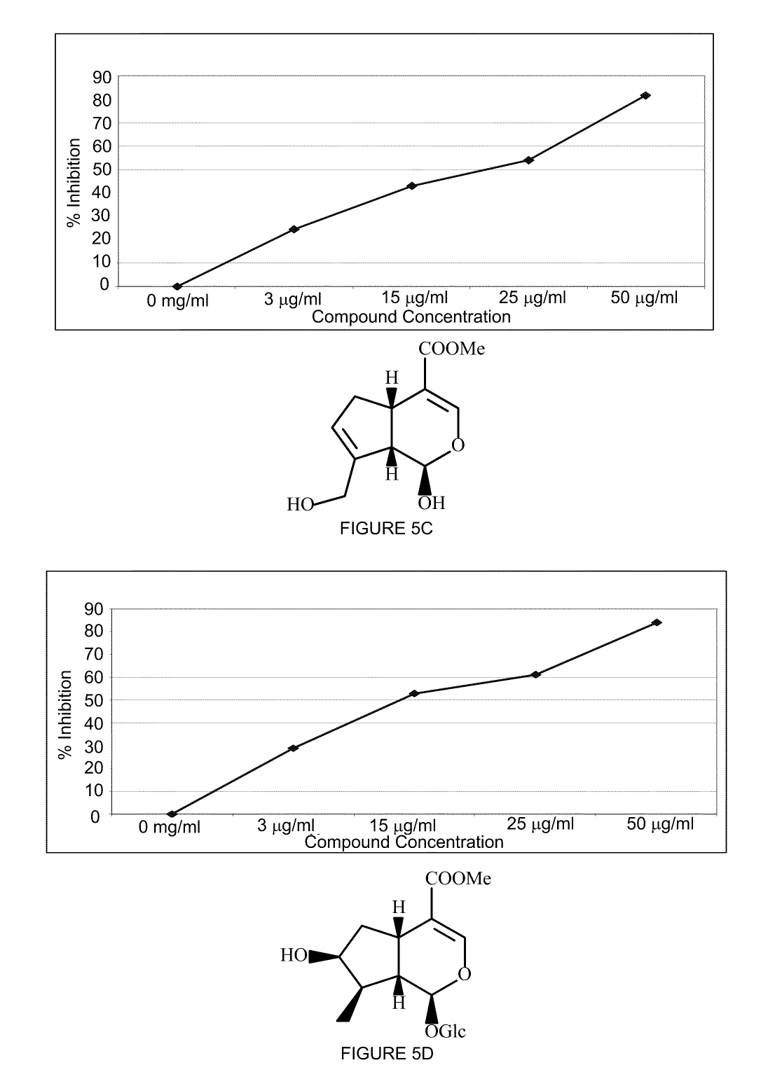

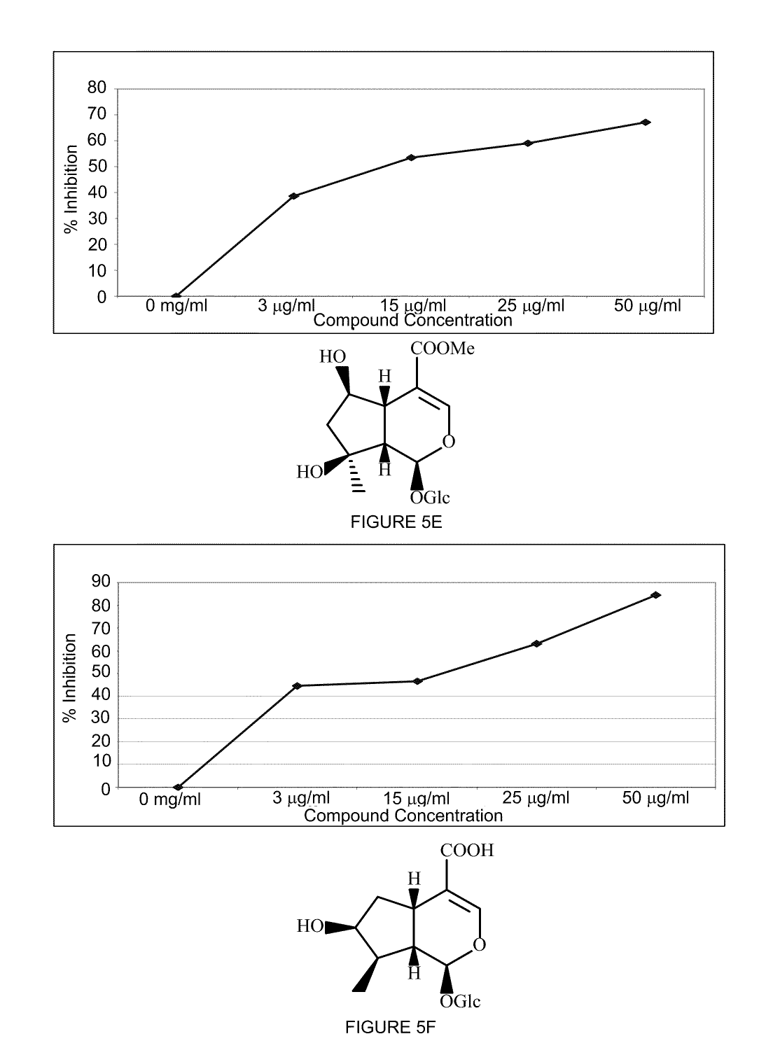

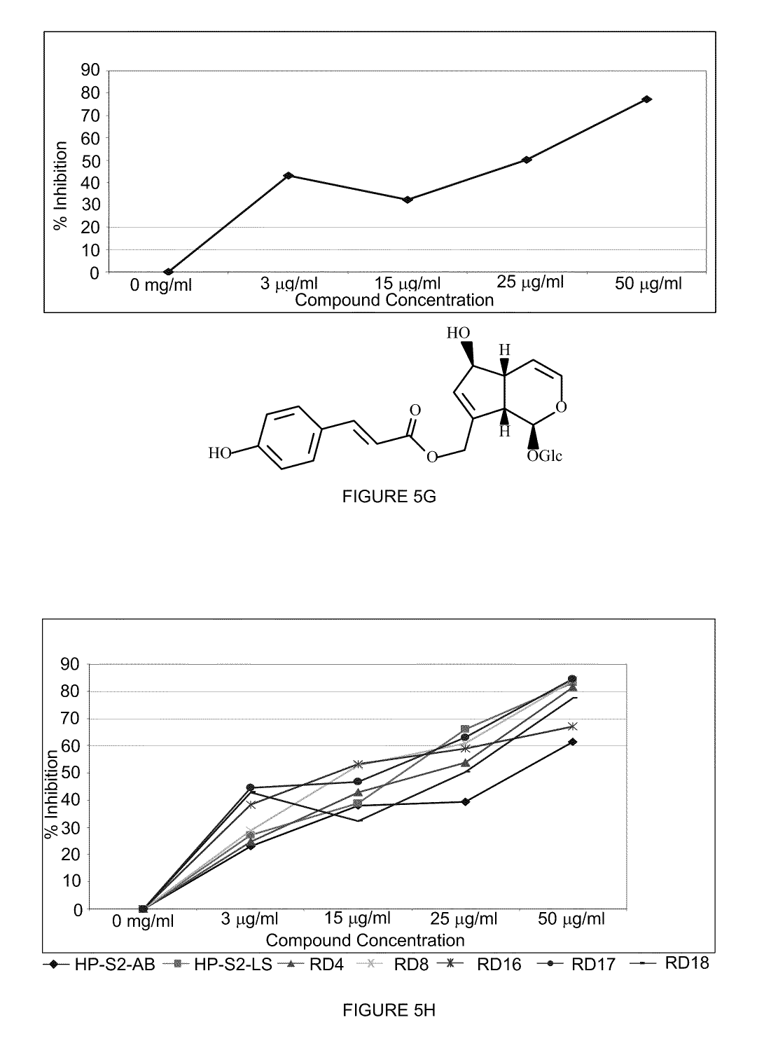

Results: Data from Huh 7 cells preincubated with compound did not show inhibition; therefore only data from virus preincubation was shown. The results suggested the active compounds act on the E1E2 protein of the HCV virus, and not on cellular components. The dose-response data were shown as “% Infectivity” to demonstrate reduction of infectivity in comparison to untreated infected or DMSO control infected. See FIGS. 5A-E.

### Experimental Protocol:

### I. Cells and Virus:

Cell Lines: Huh7 p29

Virus: HCV JFH1 wt p5d7 Huh7 B.S. 06-2007.

### II. Special Reagents:

Compounds selected: HP-S2-AB, HP-S2-LS, RD4, RD8, RD16, RD17, RD18 (all compounds are at a concentration of 5 μg/μl).

IFN-β at 50 U/μl

DMSO

### III. Methodology:

1. Seed plates at 5000 cells/well. Incubate for 24 hours at 37° C., 5% CO2.
2. Each well was infected with 100 ffu of JFH1 wt virus.
7 ffu/μl virus stock concentration→for a 100 ffu→14.3 μl of virus stock add 85.7 μl of cDMEM
There approximately 200 wells infected, therefore: 200 wells×14.3 μl of virus stock/well=2.86 ml; 200 wells×85.711 of cDMEM/well=17.14 ml. This was the stock virus used for the experiment.
3. The compound stocks at 5 μg/μl were diluted as follows:
[compound] of 3 μg/ml—100× dilution of stock→add 6 μl of 100× stock/well
[compound] of 15 μg/ml—10× dilution of stock→add 3 μl of 10× stock/well
[compound] of 25 μg/ml—10× dilution of stock→add 5 μl of 10× stock/well
[compound] of 50 μg/ml—1× dilution of stock→add 1 μl of 1× stock/well
4. For the plate that will be pretreated with the compound:
a. For each triplicate prepare a 1.5 ml tube by adding appropriate amount of cDMEM and compound for 3 wells (320 μl):

[compound] of 3 μg/ml—add 19.2 μl of 100× drug+300.8 μl of cDMEM

[compound] of 15 μg/ml—add 9.6 μl of 10× drug+310.4 μl of cDMEM

[compound] of 25 μg/ml—add 16 μl of 10× drug+304 μl of cDMEM

[compound] of 50 μg/ml—add 3.2 μl of 1× drug+316.8 μl of cDMEM

b. Added 100 μl of above mixtures to designated wells
c. Incubated 96 well plate for 3 hours with compound at 37° C. 5% CO₂.
d. Removed compound and wash each well 3× with chilled PBS.
e. Added 100 μl of viral inoculum and incubate at 37° C. 5% CO₂.
f. 24 hours PI add 100 μl of 0.5% methyl cellulose to each well for a final methyl cellulose concentration of 0.25%.
g. Fixed and stained 48 hours PI.
5. For the plate being infected with virus treated with compound:
a. For each triplicate prepare a 1.5 ml tube by adding appropriate amount of viral inoculum and drug for 3 wells (320 μl):

[compound] of 3 μg/ml—add 1.92 μl of 10× drug+320 μl of viral inoculum

[compound] of 15 μg/ml—add 9.6 μl of 10× drug+320 μl of viral inoculum

[compound] of 25 μg/ml—add 16 μl of 10× drug+320 μl of viral inoculum

[compound] of 50 μg/ml—add 3.2 μl of 1× drug+320 μl of viral inoculum

b. Added 100 μl of above mixtures to designated wells
c. Incubate 96 well plate at 37° C. 5% CO₂.
d. 24 hours PI add 100 μl of 0.5% methyl cellulose to each well for a final methyl cellulose concentration of 0.25%.
e. Fixed and stained 48 hours PI.
6. For the IFN-β control wells, IFN-β was premixed with viral inoculum for a final concentration of 10 U/ml.
7. For DMSO control, 1.6 μl of DMSO+320 μl of cDMEM. Add 100 μl to each designated well.

The results are shown on FIGS. 5A-G. FIG. 5A shows the inhibition of HCV JFH1 infectivity by lamiridosin. FIG. 5B shows the inhibition of HCV JFH1 infectivity by a lamiridosin isolated from plant source. FIG. 5C shows the inhibition of HCV JFH1 infectivity by a lamiridosin derivative RD-4. FIG. 5D shows the inhibition of HCV JFH1 infectivity by the enzyme hydrolysis product of RD8, wherein RD8 was incubated with glucosidase. FIG. 5E shows the inhibition of HCV JFH1 infectivity by the enzyme hydrolysis product of RD16, wherein RD16 was incubated with glucosidase. FIG. 5F shows the inhibition of HCV JFH1 infectivity by the enzyme hydrolysis product of RD17, wherein RD17 was incubated with glucosidase. FIG. 5G shows the inhibition of HCV JFH1 infectivity by the enzyme hydrolysis product of RD18, wherein RD18 was incubated with glucosidase. FIG. 5H shows the summary of the results of 5A-G, the inhibition of HCV JFH1 infectivity. The estimated IC₅₀ of the compounds is as follows:

TABLE 8
Estimated IC₅₀

| No. | Compound | IC₅₀ |
|---|---|---|
| 1 | Lamiridosin (FIG. 5A) | 20 μg/ml |
| 2 | RD-4 | 22 μg/ml |
| 3 | RD-8 | 15 μg/ml |
| 4 | RD-16 | 14 μg/ml |
| 5 | RD 17 | 17 μg/ml |
| 6 | RD-18 | 26 μg/ml |

What this claimed is:

1. A method for inhibiting the infection or replication of HCV, comprising administering an effective amount of a composition comprising at least one of lamiridosin, a derivative of lamiridosin, or an iridoid or a combination thereof.

2. The method of claim 1, wherein the composition comprises lamiridosin,

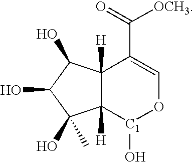

3. The method of claim 1 wherein the composition comprises a derivative of lamiridosin.

4. The method of claim 3, wherein the derivative of lamiridosin is 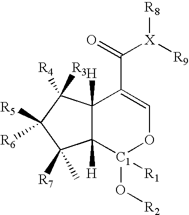
wherein R₁ is hydrogen, or R and OR₁ taken together can form an oxo group;
R₂ is hydrogen, β-D-glucopyranosyl group, (C₁₋₁₀)alkyl, (C₂₋₁₀)alkenyl, (C₂₋₁₀)alkynyl, or any of which groups can be optionally substituted with one to twenty-two of the same or different halogen atoms;
R₃ and R₄ are independently hydrogen, —OR′, —OC(O)R″, halo, R₃ and R₄ together can form an oxo group, and R′ and R″ are as described as below;
R₅ and R₆ are independently hydrogen, —OR′, —OC(O)R″, halo, R₅ and R₆ together can form an oxo group, and R′ and R″ are as described as below;
R₇ is hydrogen or hydroxy;
R₈ and R₉ are independently hydrogen, (C₁₋₁₀)alkyl, (C₂₋₁₀)alkenyl, (C₂₋₁₀)alkynyl, or any of which groups can be optionally substituted with one to twenty-two of the same or different halogen atoms;
R′ is hydrogen, (C₁₋₁₀)alkyl, (C₂₋₁₀)alkenyl, (C₂₋₁₀)alkynyl, or any of which groups can be optionally substituted with one to twenty-two of the same or different halogen atoms;
R″ is hydrogen, (C₁₋₁₀)alkyl, (C₂₋₁₀)alkenyl, (C₂₋₁₀)alkynyl, (C₃₋₁₀)cycloalkyl, (C₈₋₁₄)bicycloalkyl, (C₈₋₁₄)tricycloalkyl, (C₄₋₁₀)cycloalkenyl, (C₈₋₁₄)bicycloalkenyl, (C₈₋₁₄)tricycloalkenyl, aryl, substituted aryl, (3- to 6-membered)heterocycle, or any of which groups can be optionally substituted with one to twenty-two of the same or different halogen atoms; and
X is oxygen or nitrogen.

5. The method of claim 3, wherein the derivative is 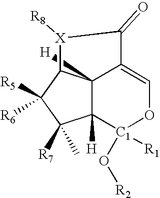
wherein R₁ is hydrogen, or R and OR₁ taken together can form an oxo group;
R₂ is hydrogen, β-D-glucopyranosyl group, (C₁₋₁₀)alkyl, (C₂₋₁₀)alkenyl, (C₂₋₁₀)alkynyl, or any of which groups can be optionally substituted with one to twenty-two of the same or different halogen atoms;
R₅ and R₆ are independently hydrogen, —OR′, —OC(O)R″, halo, R₅ and R₆ together can form an oxo group, and R′ and R″ are as described as below;
R₈ is hydrogen, (C₁₋₁₀)alkyl, (C₂₋₁₀)alkenyl, (C₂₋₁₀)alkynyl, or any of which groups can be optionally substituted with one to twenty-two of the same or different halogen atoms;
X is oxygen or nitrogen;
R′ is hydrogen, (C₁₋₁₀)alkyl, (C₂₋₁₀)alkenyl, (C₂₋₁₀)alkynyl, or any of which groups can be optionally substituted with one to twenty-two of the same or different halogen atoms;
R″ is hydrogen, (C₁₋₁₀)alkyl, (C₂₋₁₀)alkenyl, (C₂₋₁₀)alkynyl, (C₃₋₁₀)cycloalkyl, (C₈₋₁₄)bicycloalkyl, (C₈₋₁₄)tricycloalkyl, (C₄₋₁₀)cycloalkenyl, (C₈₋₁₄)bicycloalkenyl, (C₈₋₁₄)tricycloalkenyl, aryl, substituted aryl, (3- to 6-membered)heterocycle, or any of which groups can be optionally substituted with one to twenty-two of the same or different halogen atoms.

6. The method of claim 1, wherein the composition comprises at least one iridoid.

7. The method of claim 6, wherein the iridoid is aucubin, catapol, geniposide, loganinic acid, loganin, cornin, shanziside methyl ester, gardenoside, harpogoside, agnuside, picroside II, ammarogentin, sweroside, swertiamarin, oleuropein, aucubin aglycone, catapol aglycone, geniposide aglycone, loganinic acid aglycone, loganin aglycone, cornin aglycone, shanziside methyl ester aglycone, gardenoside aglycone, harpogoside aglycone, agnuside aglycone, picroside II aglycone, sweroside aglycone, swertiamarin aglycone, or oleuropein aglycone.

8. The method of claim 1, wherein the composition further comprises a pharmaceutically acceptable formulation.

9. The method of claim 1, wherein the composition further comprises at least one pharmaceutically active agent.

10. A method for inhibiting the infection or replication of HBV, comprising administering an effective amount of a composition comprising at least one of lamiridosin, a derivative of lamiridosin, or an iridoid or a combination thereof.

11. The method of claim 10, wherein the composition comprises lamiridosin,

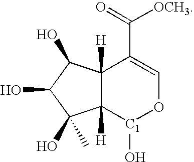

12. The method of claim 10, wherein the composition comprises a derivative of lamiridosin.

13. The method of claim 12, wherein the derivative of lamiridosin is 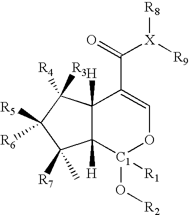
wherein R₁ is hydrogen, or R and OR₁ taken together can form an oxo group;
R₂ is hydrogen, β-D-glucopyranosyl group, (C₁₋₁₀)alkyl, (C₂₋₁₀)alkenyl, (C₂₋₁₀)alkynyl, or any of which groups can be optionally substituted with one to twenty-two of the same or different halogen atoms;
R₃ and R₄ are independently hydrogen, —OR′, —OC(O)R″, halo, R₃ and R₄ together can form an oxo group, and R′ and R″ are as described as below;
R₅ and R₆ are independently hydrogen, —OR′, —OC(O)R″, halo, R₅ and R₆ together can form an oxo group, and R′ and R″ are as described as below;
R₇ is hydrogen or hydroxy;
R₈ and R₉ are independently hydrogen, (C₁₋₁₀)alkyl, (C₂₋₁₀)alkenyl, (C₂₋₁₀)alkynyl, or any of which groups can be optionally substituted with one to twenty-two of the same or different halogen atoms;
R′ is hydrogen, (C₁₋₁₀)alkyl, (C₂₋₁₀)alkenyl, (C₂₋₁₀)alkynyl, or any of which groups can be optionally substituted with one to twenty-two of the same or different halogen atoms;
R″ is hydrogen, (C₁₋₁₀)alkyl, (C₂₋₁₀)alkenyl, (C₂₋₁₀)alkynyl, (C₃₋₁₀)cycloalkyl, (C₈₋₁₄)bicycloalkyl, (C₈₋₁₄)tricycloalkyl, (C₄₋₁₀)cycloalkenyl, (C₈₋₁₄)bicycloalkenyl, (C₈₋₁₄)tricycloalkenyl, aryl, substituted aryl, (3- to 6-membered)heterocycle, or any of which groups can be optionally substituted with one to twenty-two of the same or different halogen atoms; and
X is oxygen or nitrogen.

14. The method of claim 12, wherein the derivative is 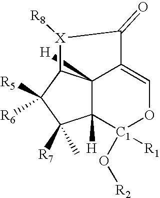
wherein R₁ is hydrogen, or R and OR₁ taken together can form an oxo group;
R₂ is hydrogen, β-D-glucopyranosyl group, (C₁₋₁₀)alkyl, (C₂₋₁₀)alkenyl, (C₂₋₁₀)alkynyl, or any of which groups can be optionally substituted with one to twenty-two of the same or different halogen atoms;
R₅ and R₆ are independently hydrogen, —OR′, —OC(O)R″, halo, R₅ and R₆ together can form an oxo group, and R′ and R″ are as described as below;
R₈ is hydrogen, (C₁₋₁₀)alkyl, (C₂₋₁₀)alkenyl, (C₂₋₁₀)alkynyl, or any of which groups can be optionally substituted with one to twenty-two of the same or different halogen atoms;
X is oxygen or nitrogen;
R′ is hydrogen, (C₁₋₁₀)alkyl, (C₂₋₁₀)alkenyl, (C₂₋₁₀)alkynyl, or any of which groups can be optionally substituted with one to twenty-two of the same or different halogen atoms;
R″ is hydrogen, (C₁₋₁₀)alkyl, (C₂₋₁₀)alkenyl, (C₂₋₁₀)alkynyl, (C₃₋₁₀)cycloalkyl, (C₈₋₁₄)bicycloalkyl, (C₈₋₁₄)tricycloalkyl, (C₄₋₁₀)cycloalkenyl, (C₈₋₁₄)bicycloalkenyl, (C₈₋₁₄)tricycloalkenyl, aryl, substituted aryl, (3- to 6-membered)heterocycle, or any of which groups can be optionally substituted with one to twenty-two of the same or different halogen atoms.

15. The method of claim 10, wherein the composition comprises at least one iridoid.

16. The method of claim 15, wherein the iridoid is aucubin, catapol, geniposide, loganinic acid, loganin, cornin, shanziside methyl ester, gardenoside, harpogoside, agnuside, picroside II, armarogentin, sweroside, swertiamarin, oleuropein, aucubin aglycone, catapol aglycone, geniposide aglycone, loganinic acid aglycone, loganin aglycone, cornin aglycone, shanziside methyl ester aglycone, gardenoside aglycone, harpogoside aglycone, agnuside aglycone, picroside II aglycone, sweroside aglycone, swertiamarin aglycone, or oleuropein aglycone.

17. The method of claim 10, wherein the composition further comprises at least one pharmaceutically active agent.

18. A compound, wherein the compound is a derivative of lamiridosin.

19. The compound of 15, wherein the compound is 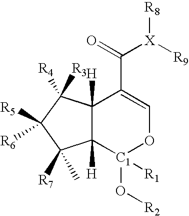
wherein R₁ is hydrogen, or R and OR₁ taken together can form an oxo group;
R₂ is hydrogen, β-D-glucopyranosyl group, (C₁₋₁₀)alkyl, (C₂₋₁₀)alkenyl, (C₂₋₁₀)alkynyl, or any of which groups can be optionally substituted with one to twenty-two of the same or different halogen atoms;
R₃ and R₄ are independently hydrogen, —OR′, —OC(O)R″, halo, R₃ and R₄ together can form an oxo group, and R′ and R″ are as described as below;
R₅ and R₆ are independently hydrogen, —OR′, —OC(O)R″, halo, R₅ and R₆ together can form an oxo group, and R′ and R″ are as described as below;
R₇ is hydrogen or hydroxy;
R₈ and R₉ are independently hydrogen, (C₁₋₁₀)alkyl, (C₂₋₁₀)alkenyl, (C₂₋₁₀)alkynyl, or any of which groups can be optionally substituted with one to twenty-two of the same or different halogen atoms;
R′ is hydrogen, (C₁₋₁₀)alkyl, (C₂₋₁₀)alkenyl, (C₂₋₁₀)alkynyl, or any of which groups can be optionally substituted with one to twenty-two of the same or different halogen atoms;
R″ is hydrogen, (C₁₋₁₀)alkyl, (C₂₋₁₀)alkenyl, (C₂₋₁₀)alkynyl, (C₃₋₁₀)cycloalkyl, (C₈₋₁₄)bicycloalkyl, (C₈₋₁₄)tricycloalkyl, (C₄₋₁₀)cycloalkenyl, (C₈₋₁₄)bicycloalkenyl, (C₈₋₁₄)tricycloalkenyl, aryl, substituted aryl, (3- to 6-membered)heterocycle, or any of which groups can be optionally substituted with one to twenty-two of the same or different halogen atoms; and
X is oxygen or nitrogen.

20. The compound of claim 15, wherein the compound is 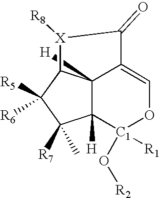
wherein R₁ is hydrogen, or R and OR₁ taken together can form an oxo group;
R₂ is hydrogen, β-D-glucopyranosyl group, (C₁₋₁₀)alkyl, (C₂₋₁₀)alkenyl, (C₂₋₁₀)alkynyl, or any of which groups can be optionally substituted with one to twenty-two of the same or different halogen atoms;
R₅ and R₆ are independently hydrogen, —OR′, —OC(O)R″, halo, R₅ and R₆ together can form an oxo group, and R′ and R″ are as described as below;
R₈ is hydrogen, (C₁₋₁₀)alkyl, (C₂₋₁₀)alkenyl, (C₂₋₁₀)alkynyl, or any of which groups can be optionally substituted with one to twenty-two of the same or different halogen atoms;
X is oxygen or nitrogen;
R′ is hydrogen, (C₁₋₁₀)alkyl, (C₂₋₁₀)alkenyl, (C₂₋₁₀)alkynyl, or any of which groups can be optionally substituted with one to twenty-two of the same or different halogen atoms;
R″ is hydrogen, (C₁₋₁₀)alkyl, (C₂₋₁₀)alkenyl, (C₂₋₁₀)alkynyl, (C₃₋₁₀)cycloalkyl, (C₈₋₁₄)bicycloalkyl, (C₈₋₁₄)tricycloalkyl, (C₄₋₁₀)cycloalkenyl, (C₈₋₁₄)bicycloalkenyl, (C₈₋₁₄)tricycloalkenyl, aryl, substituted aryl, (3- to 6-membered)heterocycle, or any of which groups can be optionally substituted with one to twenty-two of the same or different halogen atoms.


SEQUENCE LISTING (3 sequences)

SEQ ID NO: 1 - DNA, 22 bp, Artificial Sequence
  Hepatitis B Virus Probe Sequence
tgtgtctgcggcgttttatcat

SEQ ID NO: 2 - DNA, 18 bp, Artificial Sequence
  Hepatitis B Virus Primer Sequence
tgtcctggttatcgctgg

SEQ ID NO: 3 - DNA, 19 bp, Artificial Sequence
  Hepatitis B Virus Primer Sequence
caaacgggcaacatacctt